(19) United States
(12) Patent Application Publication
(10) Pub. No.: US 2015/0157590 A9
(43) Pub. Date: Jun. 11, 2015

(54) COCRYSTALS OF (N,N-DIETHYLCARBAMOYL)METHYL METHYL (2E)BUT-2-ENE-1,4-DIOATE

(71) Applicant: XenoPort, Inc., Santa Clara, CA (US)

(72) Inventors: Chen Mao, Mountain View, CA (US); Scott L. Childs, Atlanta, GA (US)

(73) Assignee: XenoPort, Inc., Santa Clara, CA (US)

(21) Appl. No.: 14/072,138

(22) Filed: Nov. 5, 2013

Related U.S. Application Data
(60) Provisional application No. 61/722,413, filed on Nov. 5, 2012.

(51) Int. Cl. A61K 31/225; A61K 31/194; A61K 31/17

(52) U.S. Cl. CPC A61K 31/225; A61K 31/17; A61K 31/194

(57) ABSTRACT

Disclosed herein are cocrystals of (N,N-Diethylcarbamoyl)methyl methyl (2E)but-2-ene-1,4-dioate, which is a prodrug of methyl hydrogen fumarate.

### CROSS-REFERENCE TO RELATED APPLICATIONS

This application claims the benefit under 35 U.S.C. §119(e) of U.S. Provisional Application Ser. No. 61/722,413, filed Nov. 5, 2012, the contents of which are incorporated herein by reference in its entirety. Reference is made to U.S. patent application Ser. Nos. 13/973,456, filed Aug. 22, 2013: 13/973.622, filed Aug. 22, 2013; and 13/973,542, filed Aug. 22, 2013; the contents of each of which are incorporated herein by reference in their entirety.

### FIELD

Disclosed herein are novel cocrystalline forms of a prodrug of methyl hydrogen fumarate, also known as monomethyl fumarate.

### BACKGROUND

The compound (N,N-Diethylcarbamoyl)methyl methyl (2E)but-2-ene-1,4-dioate has the following chemical structure:

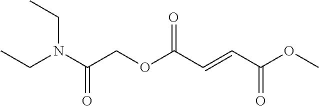 [SMILES: CCN(CC)C(=O)COC(=O)/C=C/C(=O)OC]

This compound was synthesized in Example 1 of Gangakhedkar et al., U.S. Pat. No. 8,148,414. The compound is a prodrug of methyl hydrogen fumarate (MHF) and has a disclosed melting point of between 53° C. and 56° C.

Cocrystals are crystals that contain two or more non-identical molecules that form a crystalline structure. The intermolecular interactions between the non-identical molecules in the resulting crystal structures can result in physical and chemical properties that differ from the properties of the individual components. Such properties can include, for example, melting point, solubility, chemical stability, mechanical properties and others. Examples of cocrystals may be found in the Cambridge Structural database and in Etter, et al., “The use of cocrystallization as a method of studying hydrogen bond preferences of 2-aminopyridine” J. Chem. Soc., Chem. Commun. (1990), 589-501; Etter, et al., “Graph-set analysis of hydrogen-bond patterns in organic crystals” Acta Crystallogr., Sect. B, Struct. Sci. (1990), B46; 256-262; and Etter, et al., “Hydrogen bond directed cocrystallization and molecular recognition properties of diarylureas” J. Am. Chem. Soc. (1990), 112; 8415-8426. Additional information relating to cocrystals can be found in: Carl Henrik Gorbotz and Hans-Petter Hersleth, “On the inclusion of solvent molecules in the crystal structures of organic compounds”; Acta Cryst. (2000), B625-534; and Senthil Kumar, et al., “Molecular Complexes of Some Mono- and Dicarboxylic Acids with trans-1,4-Dithiane-1,4-dioxide” American Chemical Society, Crystal Growth & Design (2002), 2(4). 313-318.

### SUMMARY

The present disclosure describes cocrystalline forms of (N,N-Diethylcarbamoyl)methyl methyl (2E)but-2-ene-1,4-dioate having improved physicochemical properties that may be used in pharmaceutical processing, in pharmaceutical compositions and in therapeutic methods of treatment.

In a first aspect, a cocrystal of (N,N-Diethylcarbamoyl)methyl methyl (2E)but-2-ene-1,4-dioate and urea, pharmaceutical compositions containing the cocrystal, and methods of administering the cocrystal to a patient for treating a disease, are provided.

In a second aspect, a cocrystal of (N,N-Diethylcarbamoyl)methyl methyl (2E)but-2-ene-1,4-dioate and fumaric acid, pharmaceutical compositions containing the cocrystal, and methods of administering the cocrystal to a patient for treating a disease, are provided.

In a third aspect, a cocrystal of (N,N-Diethylcarbamoyl)methyl methyl (2E)but-2-ene-1,4-dioate and succinic acid, pharmaceutical compositions containing the cocrystal and methods of administering the cocrystal to a patient for treating a disease, are provided.

In a fourth aspect, a cocrystal of (N,N-Diethylcarbamoyl)methyl methyl (2E)but-2-ene-1,4-dioate and maleic acid, pharmaceutical compositions containing the cocrystal, and methods of administering the cocrystal to a patient for treating a disease, are provided.

In a fifth aspect, a cocrystal of (N,N-Diethylcarbamoyl)methyl methyl (2E)but-2-ene-1,4-dioate and malic acid, pharmaceutical compositions containing the cocrystal, and methods of administering the cocrystal to a patient for treating a disease, are provided.

In a sixth aspect, a cocrystal of (N,N-Diethylcarbamoyl)methyl methyl (2E)but-2-ene-1,4-dioate and citric acid, pharmaceutical compositions containing the cocrystal, and methods of administering the cocrystal to a patient for treating a disease, are provided.

### BRIEF DESCRIPTION OF THE DRAWINGS

FIG. 1 is an X-ray powder diffractogram of a cocrystal of (N,N-Diethylcarbamoyl)methyl methyl (2E)but-2-ene-1,4-dioate and urea.

FIG. 2 is a spectrogram showing the NMR spectral pattern of a cocrystal of (N,N-Diethylcarbamoyl)methyl methyl (2E)but-2-ene-1,4-dioate and urea.

FIG. 3 is a differential scanning calorimetry (DSC) thermogram of a cocrystal of (N,N-Diethylcarbamoyl)methyl methyl (2E)but-2-ene-1,4-dioate and urea.

FIG. 4 is an X-ray powder diffractogram of a cocrystal of (N,N-Diethylcarbamoyl)methyl methyl (2E)but-2-ene-1,4-dioate and fumaric acid.

FIG. 5 is a spectrogram showing the NMR spectral pattern of a cocrystal of (N,N-Diethylcarbamoyl)methyl methyl (2E)but-2-ene-1,4-dioate and fumaric acid.

FIG. 8 is a differential scanning calorimetry (DSC) thermogram of a cocrystal of (N,N-Diethylcarbamoyl)methyl methyl (2E)but-2-ene-1,4-dioate and fumaric acid.

FIG. 7 is an X-ray powder diffractogram of a cocrystal of (N,N-Diethylcarbamoyl)methyl methyl (2E)but-2-ene-1,4-dioate and succinic acid.

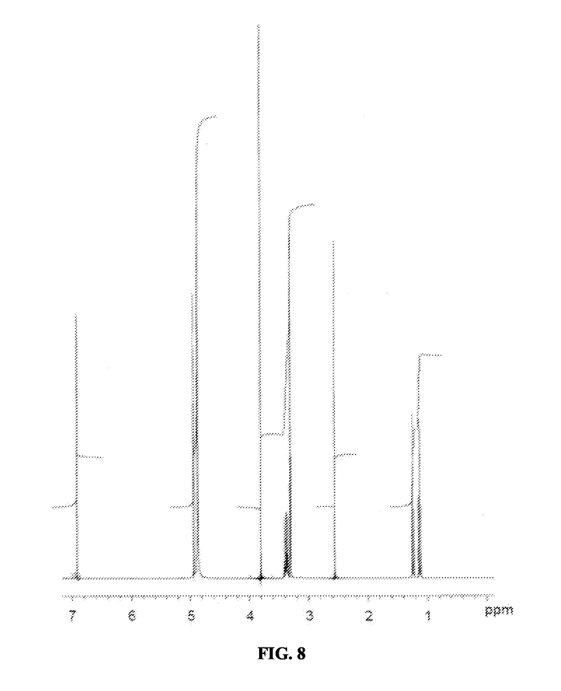

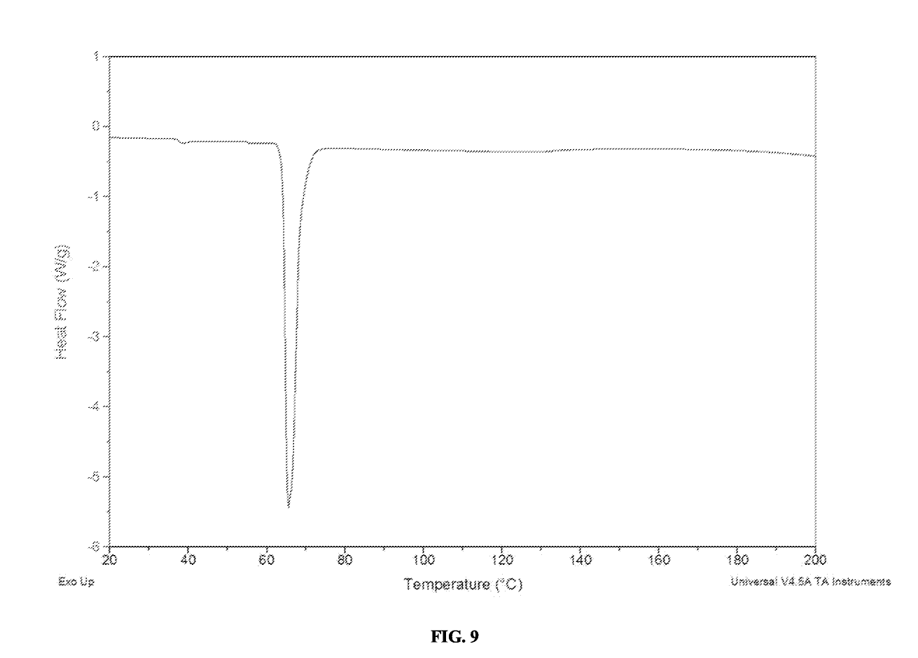

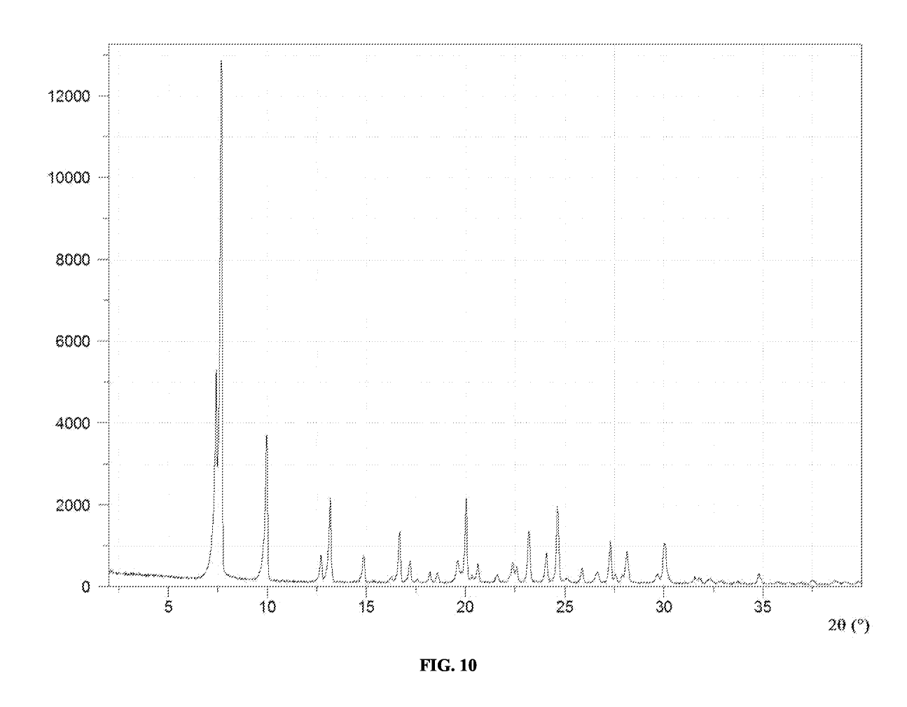

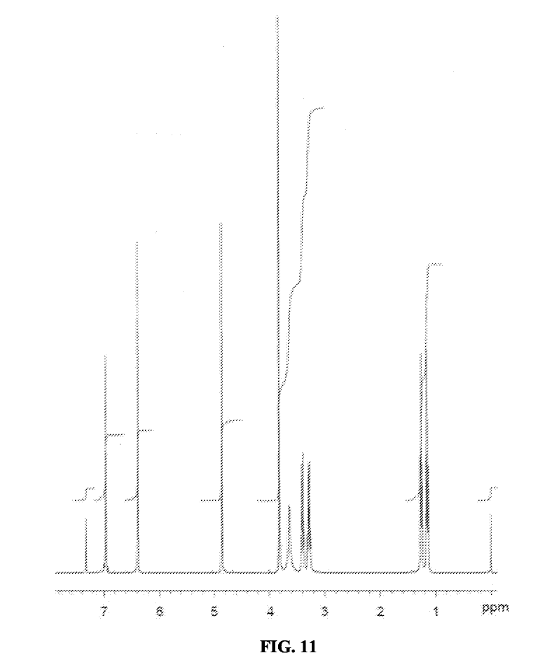

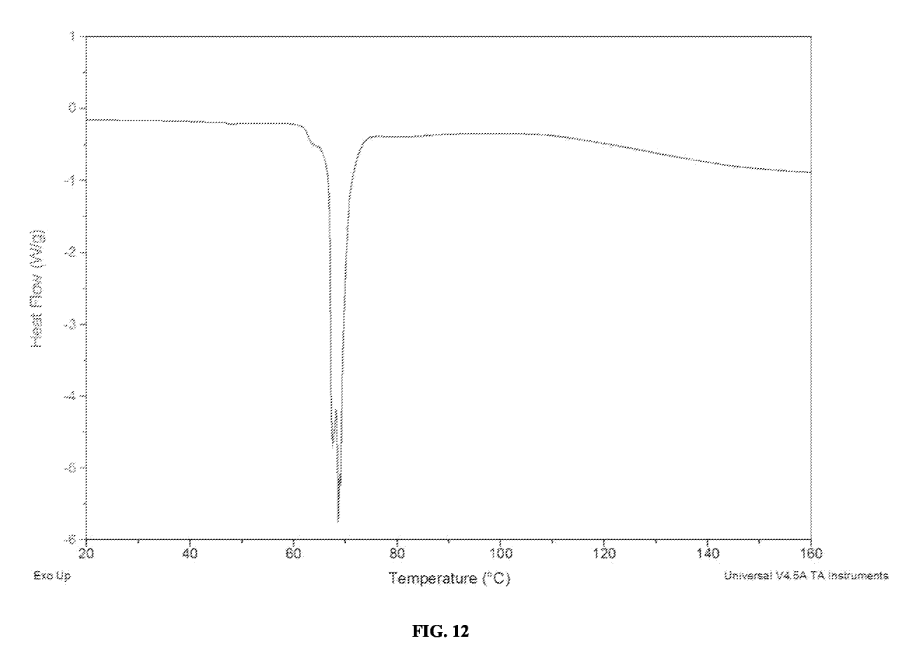

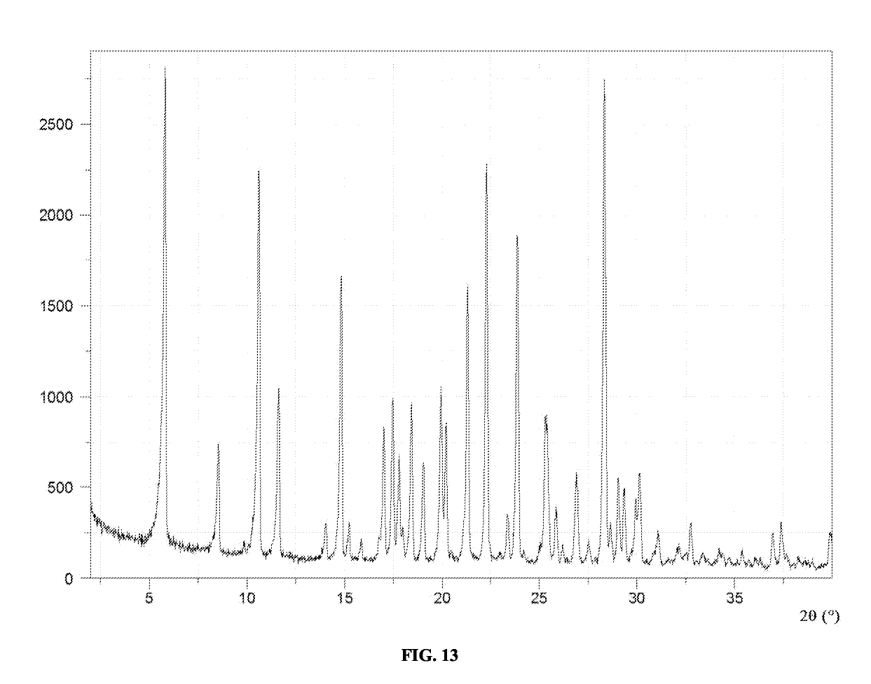

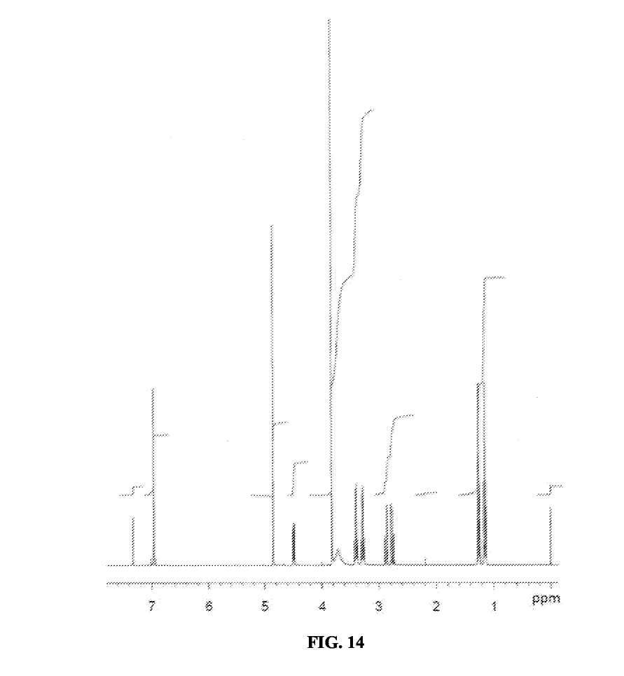

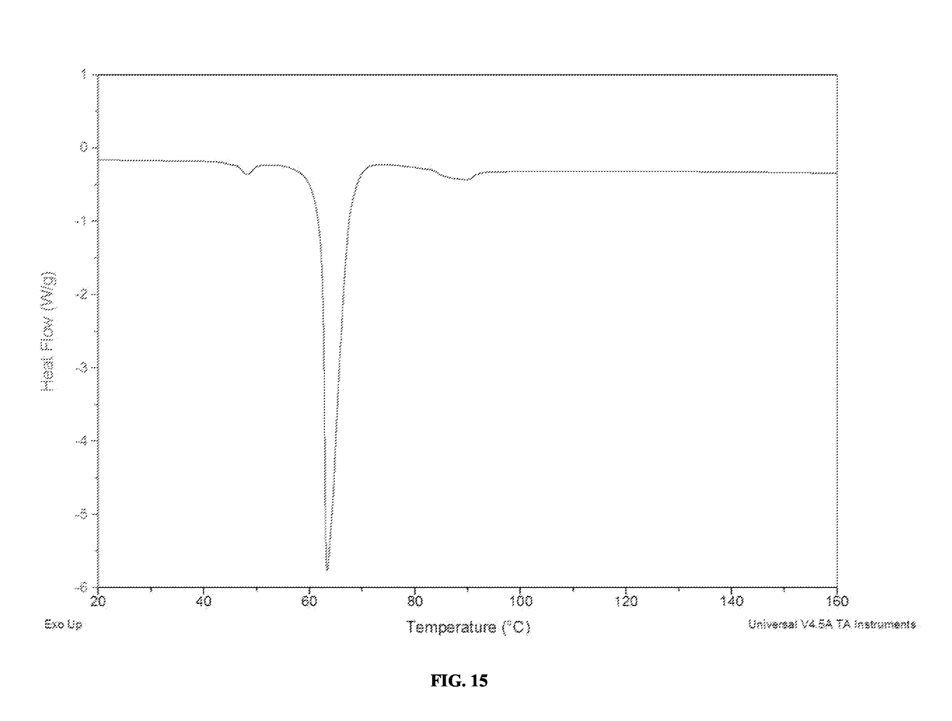

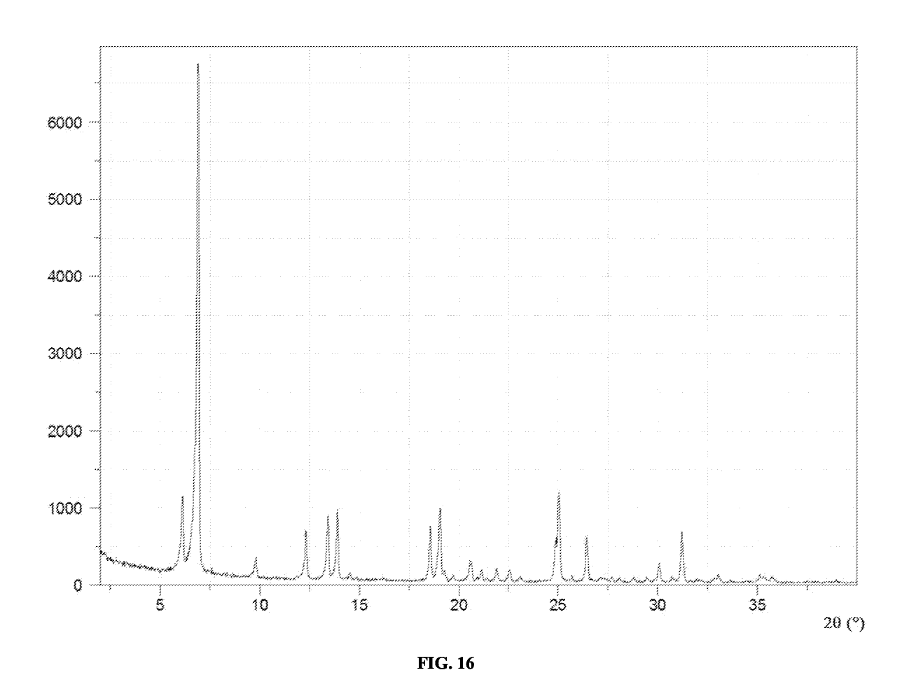

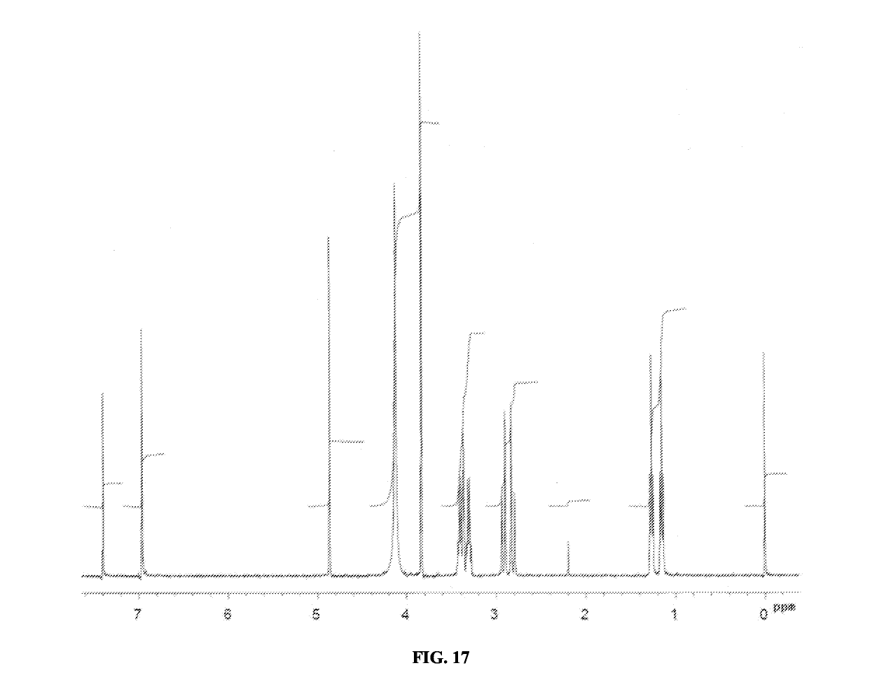

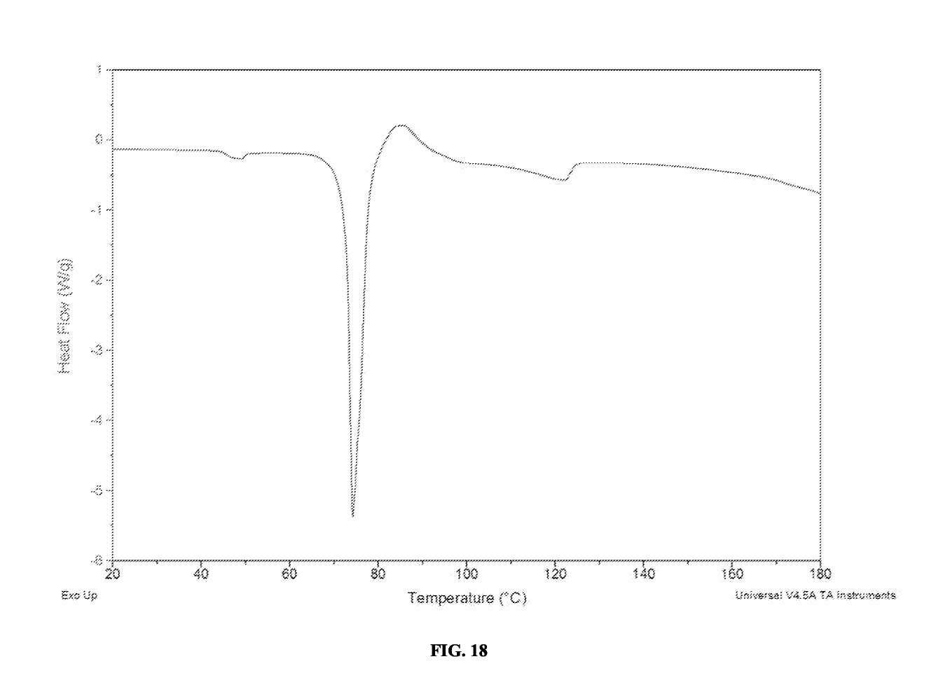

FIG. 8 is a spectrogram showing the NMR spectral pattern of a cocrystal of (N,N-Diethylcarbamoyl)methyl methyl (2E)but-2-ene-1,4-dioate and succinic acid.

FIG. 9 is a differential scanning calorimetry (DSC) thermogram of a cocrystal of (N,N-Diethylcarbamoyl)methyl methyl (2E)but-2-ene-1,4-dioate and succinic acid.

FIG. 10 is an X-ray powder diffractogram of a cocrystal of (N,N-Diethylcarbamoyl)methyl methyl (2E)but-2-ene-1,4-dioate and maleic acid.

FIG. 11 is a spectrogram showing the NMR spectral pattern of a cocrystal of (N,N-Diethylcarbamoyl)methyl methyl (2E)but-2-ene-1,4-dioate and maleic acid.

FIG. 12 is a differential scanning calorimetry (DSC) thermogram of a cocrystal of (N,N-Diethylcarbamoyl)methyl methyl (2E)but-2-ene-1,4-dioate and maleic acid.

FIG. 13 is an X-ray powder diffractogram of a cocrystal of (N,N-Diethylcarbamoyl)methyl methyl (2E)but-2-ene-1,4-dioate and malic acid.

FIG. 14 is a spectrogram showing the NMR spectral pattern of a cocrystal of (N,N-Diethylcarbamoyl)methyl methyl (2E)but-2-ene-1,4-dioate and malic acid.

FIG. 15 is a differential scanning calorimetry (DSC) thermogram of a cocrystal of (N,N-Diethylcarbamoyl)methyl methyl (2E)but-2-ene-1,4-dioate and malic acid.

FIG. 16 is an X-ray powder diffractogram of a cocrystal of (N,N-Diethylcarbamoyl)methyl methyl (2E)but-2-ene-1,4-dioate and citric acid.

FIG. 17 is a spectrogram showing the NMR spectral pattern of a cocrystal of (N,N-Diethylcarbamoyl)methyl methyl (2E)but-2-ene-1,4-dioate and citric acid.

FIG. 18 is a differential scanning calorimetry (DSC) thermogram of a cocrystal of (N,N-Diethylcarbamoyl)methyl methyl (2E)but-2-ene-1,4-dioate and citric acid.

### DETAILED DESCRIPTION

As used herein “pharmaceutically acceptable” refers to approved or approvable by a regulatory agency of the Federal government or a state government or listed in the U.S. Pharmacopoeia or other generally recognized pharmacopoeia for use in animals, and more particularly in humans.

As used herein, the terms “pharmaceutically acceptable vehicle” and “pharmaceutically acceptable carrier” refer to a pharmaceutically acceptable diluent, a pharmaceutically acceptable adjuvant a pharmaceutically acceptable excipient, or a combination of any of the foregoing, with which a composition provided by the present disclosure may be administered to a patient, which does not destroy the pharmacological activity thereof and which is non-toxic when administered in doses sufficient to provide a therapeutically effective amount of the composition.

As used herein, “treating” or “treatment” of any disease refers to reversing, alleviating, arresting, or ameliorating a disease or at least one of the clinical symptoms of a disease, reducing the risk of acquiring at least one of the clinical symptoms of a disease, inhibiting the progress of a disease or at least one of the clinical symptoms of the disease or reducing the risk of developing at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to protecting against or delaying the onset of at least one or more symptoms of a disease in a patient.

(N,N-Diethylcarbamoyl)methyl methyl (2E)but-2-ene-1,4-dioate is a prodrug of methyl hydrogen fumarate. Once administered, the compound is metabolized in vivo into an active metabolite, namely, methyl hydrogen fumarate (MHF) which is also referred to herein as monomethyl fumarate (MMF). The in vivo metabolism of (N,N-Diethylcarbamoyl)methyl methyl (2E)but-2-ene-1,4-dioate to MHF is illustrated below.

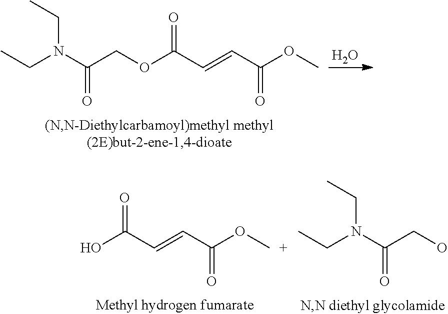

The present disclosure is directed to cocrystals of (N,N-Diethylcarbamoyl)methyl methyl (2E)but-2-ene-1,4-dioate.

By cocrystallizing the (N,N-Diethylcarbamoyl)methyl methyl (2E)but-2-ene-1,4-dioate with a guest, a new crystalline solid form is created having different properties from the (N,N-Diethylcarbamoyl)methyl methyl (2E)but-2-ene-1,4-dioate or the guest. For example, a cocrystal may have a different melting point, dissolution, solubility, hygroscopicity, bioavailability, toxicity, crystal morphology, density, loading volume, compressibility, physical stability, chemical stability, shelf life, taste, production costs, and/or manufacturing method than the crystalline prodrug.

The term “guest” refers to a compound other than (N,N-Diethylcarbamoyl)methyl methyl (2E)but-2-ene-1,4-dioate that is also a component of the cocrystal. Thus, the guest is part of the cocrystalline lattice. The guest is typically a GRAS (generally regarded as safe) compound and need not exhibit any therapeutic or pharmacological activity of its own. The Registry of Toxic Effects of Chemical Substances (RTECS) database is a useful source for toxicology information, and the GRAS list maintained by the RTECS contains about 2,500 relevant compounds that may be used in the generation of one or more cocrystals.

Six different cocrystals of (N,N-Diethylcarbamoyl)methyl methyl (2E)but-2-ene-1,4-dioate are disclosed herein. The first is a cocrystal of (N,N-Diethylcarbamoyl)methyl methyl (2E)but-2-ene-1,4-dioate and urea. The second is a cocrystal of (N,N-Diethylcarbamoyl)methyl methyl (2E)but-2-ene-1,4-dioate and fumaric acid. The third is a cocrystal of (N,N-Diethylcarbamoyl)methyl methyl (2E)but-2-ene-1,4-dioate and succinic acid. The fourth is a cocrystal of (N,N-Diethylcarbamoyl)methyl methyl (2E)but-2-ene-1,4-dioate and maleic acid. The fifth is a cocrystal of (N,N-Diethylcarbamoyl)methyl methyl (2E)but-2-ene-1,4-dioate and malic acid. The sixth is a cocrystal of (N,N-Diethylcarbamoyl)methyl methyl (2E)but-2-ene-1,4-dioate and citric acid. These six cocrystals, their melting points as well as their guest melting points and the melting point of crystalline (N,N-Diethylcarbamoyl)methyl methyl (2E)but-2-ene-1,4-dioate, are shown in Table 1.

TABLE 1

| | Crystal/ | | |
|---|---|---|---|
| | Cocrystal | | |
| | Melting Point | | Guest Melting |
| Crystal/Cocrystal | (° C.) | Guest | Point (° C.) |
| (N,N-Diethylcarbamoyl)methyl methyl | 58 ± 1 | N/A | N/A |
| (2E)but-2-ene-1,4-dioate | | | |
| (N,N-Diethylcarbamoyl)methyl methyl | 77 ± 2 | urea | 134 ± 1 |
| (2E)but-2-ene-1,4-dioate: urea | | | |
| (N,N-Diethylcarbamoyl)methyl methyl | 74 ± 2 | fumaric acid | 287 ± 1 |
| (2E)but-2-ene-1,4-dioate: fumaric acid | | | |
| (N,N-Diethylcarbamoyl)methyl methyl | 64 ± 2 | succinic acid | 185 ± 1 |
| (2E)but-2-ene-1,4-dioate: succinic acid | | | |
| (N,N-Diethylcarbamoyl)methyl methyl | 67 ± 2 | maleic acid | 133 ± 2 |
| (2E)but-2-ene-1,4-dioate: maleic acid | | | |
| (N,N-Diethylcarbamoyl)methyl methyl | 63 ± 2 | malic acid | 130 ± 1 |
| (2E)but-2-ene-1,4-dioate: malic acid | | | |
| (N,N-Diethylcarbamoyl)methyl methyl | 73 ± 2 | citric acid | 153 ± 1 |
| (2E)but-2-ene-1,4-dioate: citric acid | | | |

As can be seen from the data in Table 1, the six cocrystals disclosed herein each exhibit a higher melting point than crystalline (N,N-Diethylcarbamoyl)methyl methyl (2E)but-2-ene-1,4-dioate.

Differential scanning calorimetry, or DSC, is a thermoanalytical technique in which the difference in the amount of heat required to increase the temperature of a sample and reference is measured as a function of temperature. DSC data shows differential heat flow plotted against temperature. As a sample undergoes a thermal event, it is effectively altering the heat flow due to the latent heat associated with the thermal event, which is then reflected as a peak or a shift in baseline. DSC can be used to characterize thermal properties of cocrystals, such as melting temperature or heat of fusion. Therefore, the melting point of the six cocrystals disclosed herein can be characterized by DSC.

In addition to melting point, there are other techniques that are commonly used to identify a cocrystal. For example, the chemical identity of the components of cocrystals can often be determined with solution-state techniques such as ¹³C or ¹H NMR. However, while these solution-state techniques may help identify the prodrug and the guest, they do not provide any information about the cocrystalline solid-state structure. There are, however, several solid-state analytical techniques that can be used to provide information about solid-state structure including, for example, single crystal X-ray diffraction, powder X-ray diffraction, solid state ¹³C NMR, Raman spectroscopy, and thermal techniques.

Neither X-ray powder diffraction nor Raman spectroscopy themselves give direct data on the stoichiometry of the components which make up a cocrystal. There are techniques, however, that do provide such information. For example, single crystal X-ray diffraction gives a three-dimensional map of the atoms and bonds in the unit cell, thus directly providing the stoichiometry within the cocrystal and the precise stoichiometry within the unit cell. Solution-state techniques such as NMR may be used to confirm the molar ratios of the cocrystal component species.

Single-crystal X-ray diffraction provides three-dimensional structural information about the positions of atoms and bonds in a cocrystal. It is not always possible or feasible, however, to obtain such a structure from a cocrystal due to, for example, insufficient crystal size or difficulty in preparing crystals of sufficient quality for single-crystal X-ray diffraction. Structural identification information can, however, be obtained from other solid-state techniques such as X-ray powder diffraction and Raman spectroscopy. These techniques are used to generate data on a solid cocrystal. Once that data has been collected on a known cocrystal, that data can be used to identify the presence of that cocrystal in other materials. Thus, these data effectively characterize the cocrystal. For example, an X-ray powder diffraction pattern, or a portion thereof, can serve as a fingerprint which characterizes a cocrystal and differentiates the cocrystal from its component parts (i.e., prodrug and guest) thereby showing that the cocrystal is indeed a new material and not simply a physical mixture of the prodrug and the guest.

An X-ray powder diffraction plot is an x-y graph with scattering angles 2θ (diffraction) on the x-axis and intensity on the y-axis. The peaks within this plot can be used to characterize a cocrystal. Although the peaks within an entire diffractogram can be used to characterize a cocrystal, a subset of the more characteristic peaks can also be used to accurately characterize a cocrystal. The data is often represented by the position of the peaks on the x-axis rather than the intensity of peaks on the y-axis because peak intensity may vary with sample orientation. There is also variability in the position of peaks on the x-axis.

There are several sources of this variability, one of which comes from sample preparation. Samples of the same cocrystalline material prepared under different conditions may yield slightly different diffractograms. Factors such as particle size, moisture content, solvent content, and orientation can affect how a sample diffracts X-rays. Another source of variability comes from instrument parameters. Different X-ray instruments operate using different parameters and these may lead to slightly different diffraction patterns from the same cocrystal. Likewise, different software packages process X-ray data differently and this also leads to variability, These and other sources of variability are known to those of ordinary skill in the art.

Due to these sources of variability, it is common to recite X-ray diffraction peaks using the word “about” prior to the peak value in 2θ. The word “about” incorporates this variability which under most sampling conditions, and most data collection and data processing conditions, leads to a variability in peak position of about plus or minus 0.2 scattering angle (2θ). Thus, when a peak is said to be at about 10.5 scattering angle (2θ), under most sampling, data collection, and data processing conditions, that peak will appear anywhere between 10.3 (2θ) and 10.7 (2θ).

In characterizing the cocrystals disclosed herein, the X-ray diffraction peaks were all measured using Cu-K_{α} radiation and all peaks herein cited refer to peaks diffracted from X-rays with that wavelength. Thus, when characterizing a cocrystal, those of ordinary skill in the art will select a peak or set of peaks from the X-ray powder diffraction pattern of the cocrystal wherein at least one of those peaks is at least 0.4 (2θ) from any of the peaks in the X-ray powder diffraction patterns of the prodrug and guest of that cocrystal.

(N,N-Diethylcarbamoyl)methyl methyl (2E)but-2-ene-1,4-dioate:Urea Cocrystal

One cocrystal disclosed herein is a cocrystal of (N,N-Diethylcarbamoyl)methyl methyl (2E)but-2-ene-1,4-dioate and urea. High performance liquid chromatography (HPLC) data indicates that the stoichiometry of (N,N-Diethylcarbamoyl)methyl methyl (2E)but-2-ene-1,4-dioate to urea is 1:1. Differential scanning calorimetry (DSC) analysis of this cocrystal shows a melting point between about 75° C. and about 79° C., in certain embodiments between about 76° C. and about 78° C., and in certain embodiments at about 77° C.

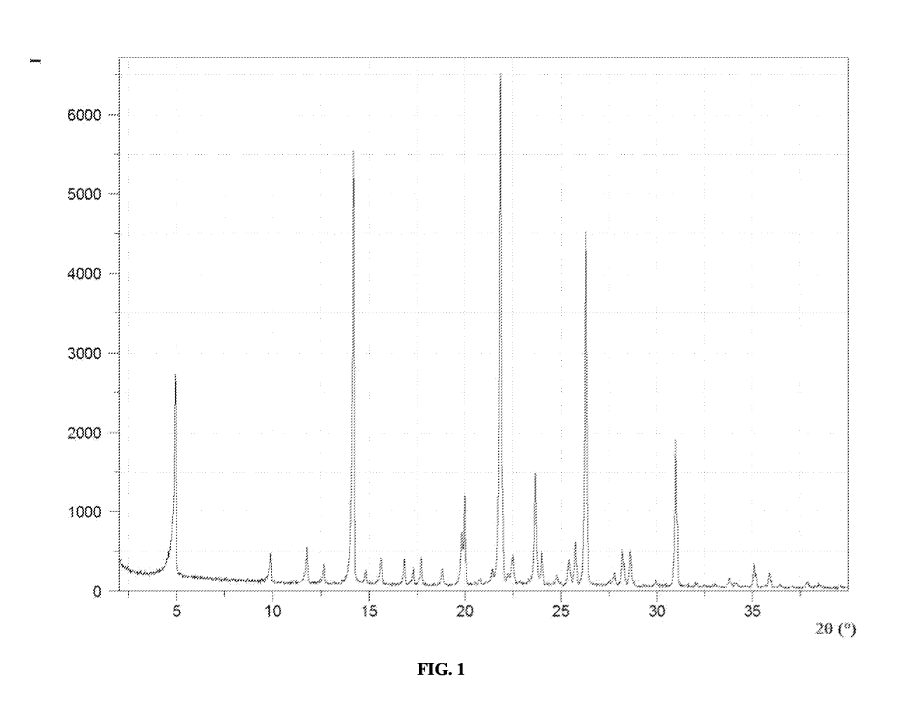

FIG. 1 is an X-ray powder diffractogram showing the diffraction pattern measured using Cu-K_{α} radiation of the (N,N-Diethylcarbamoyl)methyl methyl (2E)but-2-ene-1,4-dioate:urea cocrystal. Table 2 lists the approximate numerical values of the XRPD peak positions of the FIG. 1 diffractogram. While the entire diffractogram of FIG. 1 can be used to characterize the cocrystal, the cocrystal can also be accurately characterized with a subset of that data. For example, the XRPD peak at about 26.3 °2θ in FIG. 1 is more than 0.4 °2θ away from any XRPD peak of (N,N-Diethylcarbamoyl)methyl methyl (2E)but-2-ene-1,4-dioate. In addition, the X-ray diffraction pattern of urea shows that no urea XRPD peak occurs within 0.4 °2θ of 26.3 °2θ. Thus, the XRPD peak at 26.3 °2θ characterizes the (N,N-Diethylcarbamoyl)methyl methyl (2E)but-2-ene-1,4-dioate:urea cocrystal.

TABLE 2
XRPD Peaks for (N,N-Diethylcarbamoyl)methyl methyl
(2E)but-2-ene-1,4-dioate:urea cocrystal

| | | | | |
|---|---|---|---|---|
| Pos. | | | | |
| [°2Th.] | Height [cts] | FWHM [°2Th.] | d-spacing [Å] | Rel. Int. [%] |

Peaks (More Characteristic)

| | | | | |
|---|---|---|---|---|
| 21.8 | 6318.2 | 0.1 | 4.1 | 100.0 |
| 14.2 | 5284.0 | 0.1 | 6.2 | 83.6 |
| 26.3 | 4345.6 | 0.1 | 3.4 | 68.8 |
| 4.9 | 2503.6 | 0.1 | 17.9 | 39.6 |
| 31.0 | 1790.3 | 0.1 | 2.9 | 28.3 |

Peaks (All)

| | | | | |
|---|---|---|---|---|
| 4.9 | 2503.5 | 0.1 | 17.9 | 39.6 |
| 9.9 | 346.3 | 0.1 | 9.0 | 5.5 |
| 11.8 | 449.9 | 0.1 | 7.5 | 7.1 |
| 12.6 | 229.9 | 0.1 | 7.0 | 3.6 |
| 14.2 | 5284.0 | 0.1 | 6.2 | 83.6 |
| 14.8 | 179.3 | 0.1 | 6.0 | 2.8 |
| 15.6 | 316.4 | 0.1 | 5.7 | 5.0 |
| 16.8 | 303.9 | 0.1 | 5.3 | 4.8 |
| 17.3 | 201.8 | 0.1 | 5.1 | 3.2 |
| 17.7 | 330.4 | 0.1 | 5.0 | 5.2 |
| 18.8 | 187.4 | 0.1 | 4.7 | 3.0 |
| 19.8 | 667.0 | 0.1 | 4.5 | 10.6 |
| 20.0 | 1124.8 | 0.1 | 4.4 | 17.8 |
| 20.8 | 85.6 | 0.1 | 4.3 | 1.4 |
| 21.8 | 6318.2 | 0.1 | 4.1 | 100.0 |
| 22.5 | 358.6 | 0.1 | 4.0 | 5.7 |
| 23.7 | 1444.1 | 0.1 | 3.8 | 22.9 |
| 24.0 | 413.7 | 0.1 | 3.7 | 6.6 |
| 24.8 | 99.6 | 0.2 | 3.6 | 1.6 |
| 25.4 | 309.0 | 0.1 | 3.5 | 4.9 |
| 25.8 | 533.5 | 0.1 | 3.5 | 8.4 |
| 26.3 | 4345.6 | 0.1 | 3.4 | 68.8 |
| 27.8 | 174.4 | 0.1 | 3.2 | 2.8 |
| 28.2 | 435.4 | 0.1 | 3.2 | 6.9 |
| 28.6 | 456.4 | 0.1 | 3.1 | 7.2 |
| 29.9 | 58.3 | 0.2 | 3.0 | 0.9 |
| 31.0 | 1790.3 | 0.1 | 2.9 | 28.3 |
| 32.0 | 43.4 | 0.2 | 2.8 | 0.7 |
| 33.8 | 106.6 | 0.1 | 2.7 | 1.7 |
| 35.1 | 285.1 | 0.1 | 2.6 | 4.5 |
| 35.9 | 180.9 | 0.1 | 2.5 | 2.9 |
| 37.8 | 74.4 | 0.2 | 2.4 | 1.2 |
| 38.5 | 35.2 | 0.3 | 2.3 | 0.6 |

In another example, the XRPD peak at about 4.9 °2θ in FIG, 1 is more than 0.4 °2θ away from any peak in the X-ray powder diffraction pattern measured using Cu-K_{α} radiation of (N,N-Diethylcarbamoyl)methyl methyl (2E)but-2-ene-1,4-dioate. In addition, the X-ray diffraction pattern of urea shows that no peak occurs within 0.4 °2θ of 4.9 °2θ. Thus, the peak at 4.9 °2θ is another peak that alone or together with the peak at 26.3 °2θ characterizes the (N,N-Diethylcarbamoyl)methyl methyl (2E)but-2-ene-1,4-dioate:urea cocrystal.

Likewise, no XRPD peak of (N,N-Diethylcarbamoyl)methyl methyl (2E)but-2-ene-1,4-dioate or of urea appears within 0.4 °2θ of 11.8 °2θ in FIG. 1, which is a peak in the (N,N-Diethylcarbamoyl)methyl methyl (2E)but-2-ene-1,4-dioate;urea cocrystal. Thus, the peak at 11.8 °2θ is another peak that alone or together with the peaks at 26.3 °2θ and/or 4.9 °2θ characterizes the (N,N-Diethylcarbamoyl)methyl methyl (2E)but-2-ene-1,4-dioate:urea cocrystal.

Also, no XRPD peak of (N,N-Diethylcarbamoyl)methyl methyl (2E)but-2-ene-1,4-dioate or of urea appears within 0.4 °2θ of 28.2 √2θ in FIG. 1, which is a peak in the (N,N-Diethylcarbamoyl)methyl methyl (2E)but-2-ene-1,4-dioate:urea cocrystal. Thus, the peak at 28.2 °2θ is another peak that alone or together with the peaks at 26.3 °2θ, 4.9 °2∂ and/or 11.8 °2θ characterizes the (N,N-Diethylcarbamoyl)methyl methyl (2E)but-2-ene-1,4-dioate:urea cocrystal.

Furthermore, no XRPD peak of (N,N-Diethylcarbamoyl)methyl methyl (2E)but-2-ene-1,4-dioate or any of the urea diffraction patterns appears within 0.4 °2θ of 24.0 °2θ in FIG. 1, which is a peak in the (N,N-Diethylcarbamoyl)methyl methyl (2E)but-2-ene-1,4-dioate:urea cocrystal. Thus, the peak at 24.0 °2θ is another peak that alone or together with the peaks at 26.3 °2θ, 4.9 °2θ, 11.8 °2θ, and/or 28.2 °2θ characterizes the (N,N-Diethylcarbamoyl)methyl methyl (2E)but-2-ene-1,4-dioate:urea cocrystal.

In certain embodiments, the (N,N-Diethylcarbamoyl)methyl methyl (2E)but-2-ene-1,4-dioate:urea cocrystal exhibits characteristic scattering angles (2θ) at least at 4.9±0.2°, 14.2±0.2°, 21.8±0.2°, 26.3±0.2°, and 31.0±0.2° in an X-ray powder diffraction pattern measured using Cu-K_{α} radiation.

In certain embodiments, the (N,N-Diethylcarbamoyl)methyl methyl (2E)but-2-ene-1,4-dioate:urea cocrystal exhibits characteristic scattering angles (2θ) at least at 4.9±0.2°, 14.2±0.2°, 19.8±0.2°, 20.0±0.2°, 21.8±0.2°, 23.7±0.2°, 25.8±0.2°, 26.3±0.2°, 28.6±0.2°, and 31.0±0.2° in an X-ray powder diffraction pattern measured using Cu-K_{α} radiation.

In certain embodiments, the (N,N-Diethylcarbamoyl)methyl methyl (2E)but-2-ene-1,4-dioate:urea cocrystal exhibits characteristic scattering angles (2θ) at least at 4.9±0.2°, 9.9±0.2°, 11.8±0.2°, 14.2±0.2°, 19.8±0.2°, 20.0±0.2°, 21.8±0.2°, 22.5±0.2°, 23.7±0.2°, 24.0±0.2°, 25.8±0.2°, 26.3±0.2°, 28.2±0.2°, 28.6±0.2°, and 31.0±0.2° in an X-ray powder diffraction pattern measured using Cu-K_{α} radiation.

In certain embodiments, the (N,N-Diethylcarbamoyl)methyl methyl (2E)but-2-ene-1,4-dioate:urea cocrystal exhibits characteristic scattering angles (2θ) at least at 4.9±0.1°, 14.2±0.1°, 21.8±0.1°, 26.3±0.1°, and 31.0±0.1° in an X-ray powder diffraction pattern measured using Cu-K_{α} radiation.

In certain embodiments, the (N,N-Diethylcarbamoyl)methyl methyl (2E)but-2-ene-1,4-dioate:urea cocrystal exhibits characteristic scattering angles (2θ) at least at 4.9±0.1°, 14.2±0.1°, 19.8±0.1°, 20.0±0.1°, 21.8±0.1°, 23.7±0.1°, 25.8±0.1°, 28.3±0.1°, 28.6±0.1°, and 31.0±0.1° in an X-ray powder diffraction pattern measured using Cu-K_{α} radiation.

In certain embodiments, the (N,N-Diethylcarbamoyl)methyl methyl (2E)but-2-ene-1,4-dioate:urea cocrystal exhibits characteristic scattering angles (2θ) at least at 4.9±0.1°, 9.9±0.1°, 11.8±0.1°, 14.2±0.1°, 19.8±0.1°, 20.0±0.1°, 21.8±0.1°, 22.5±0.1°, 23.7±0.1°, 24.0±0.1°, 25.8±0.1°, 28.3±0.1°, 28.2±0.1°, 28.6±0.1° and 31.0±0.1° in an X-ray powder diffraction pattern measured using Cu-K_{α} radiation.

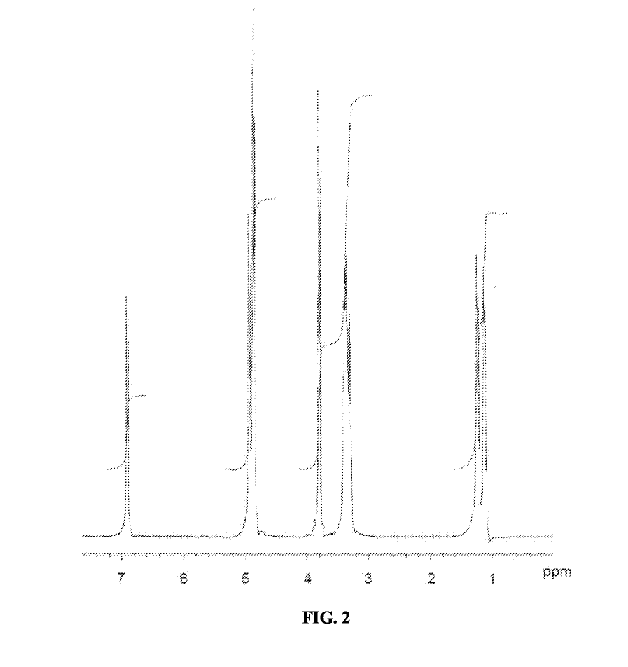

FIG. 2 is a spectrogram showing the NMR spectrum of the (N,N-Diethylcarbamoyl)methyl methyl (2E)but-2-ene-1,4-dioate:urea cocrystal. The NMR spectral pattern indicates ¹H NMR (MeOH-d₃, 400 MHz): δ 6.99-6.90 (m, 2H), 4.93 (s, 2H), 3.80 (s, 3H), 3.39 (m, Hz, 4H), 1.24 (t, J=7.2 Hz, 3H), 1.12 (t, J=7.2 Hz, 3H) for the (N,N-Diethylcarbamoyl)methyl methyl (2E)but-2-ene-1,4-dioate:urea cocrystal.

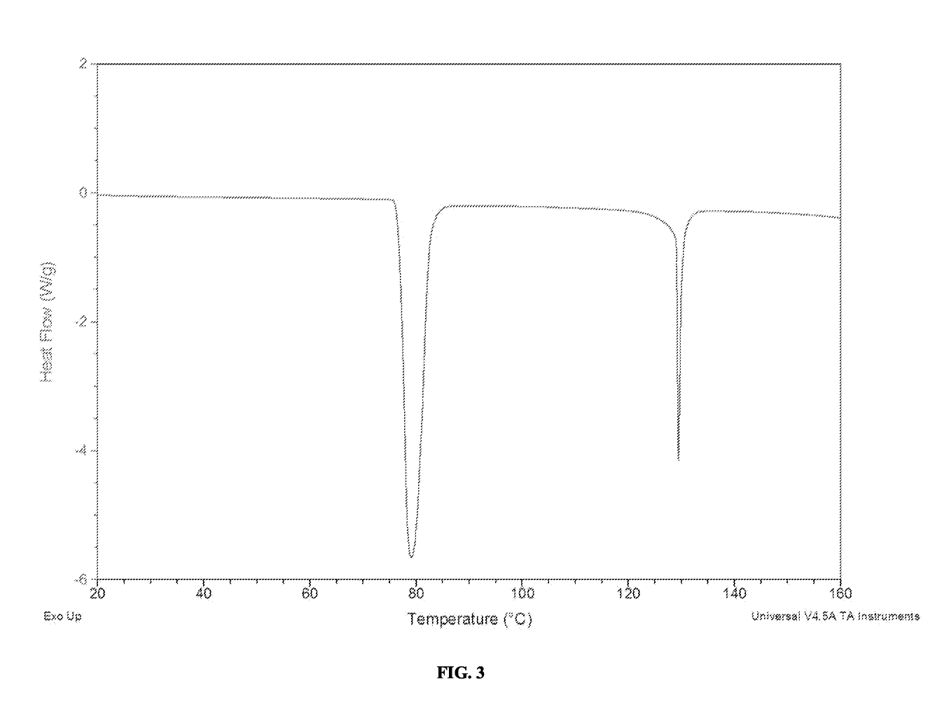

FIG. 3 is a DSC thermogram of the (N,N-Diethylcarbamoyl)methyl methyl (2E)but-2-ene-1,4-dioate:urea cocrystal. The thermogram shows the cocrystal has a melting point of about 77° C. Hot-stage microscopy study shows that the melting of the cocrystal and the crystallization of urea occurs simultaneously. The second melting transition with onset temperature at 129° C. corresponds roughly to the melting point of urea, which has a melting point of about 133° C.-135° C.

The (N,N-Diethylcarbamoyl)methyl methyl (2E)but-2-ene-1,4-dioate:urea cocrystal is expected to have a good toxicology profile, since urea is known to be safe and appears on the GRAS list from the U.S. Food and Drug Administration.

(N,N-Diethylcarbamoyl)methyl methyl (2E)but-2-ene-1,4-dioate:Fumaric Acid Cocrystal

Another cocrystal disclosed herein is a cocrystal of (N,N-Diethylcarbamoyl)methyl methyl (2E)but-2-ene-1,4-dioate and fumaric acid. HPLC data indicates that the stoichiometry of (N,N-Diethylcarbamoyl)methyl methyl (2E)but-2-ene-1,4-dioate to fumaric acid is 2:1. Differential scanning calorimetry (DSC) analysis of this cocrystal shows a melting point between about 72° C. and about 78° C., in certain embodiments between about 73° C. and about 75° C., and in certain embodiments at about 74° C.

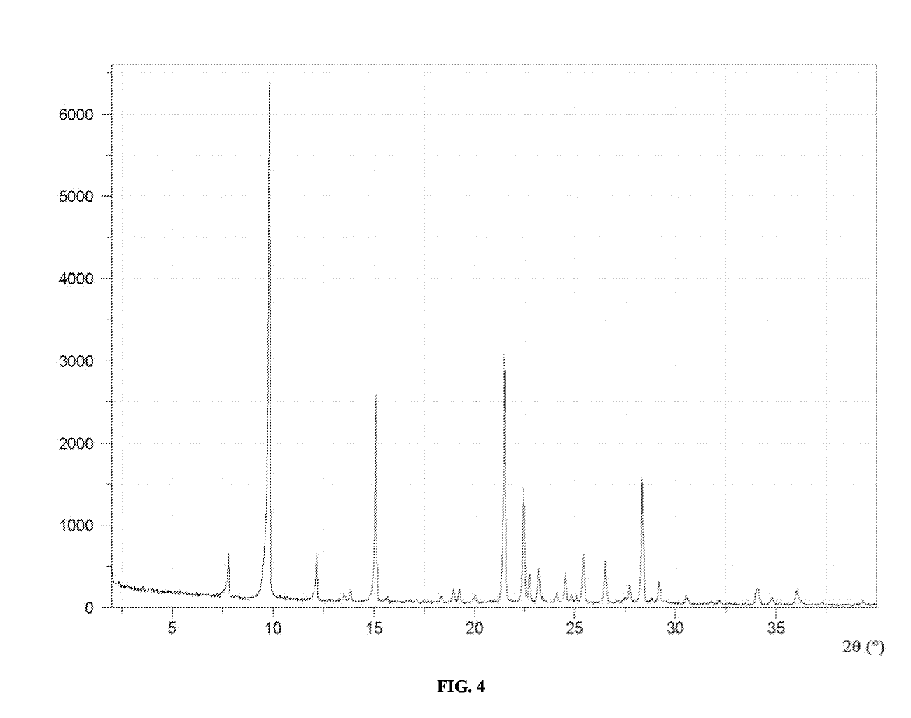

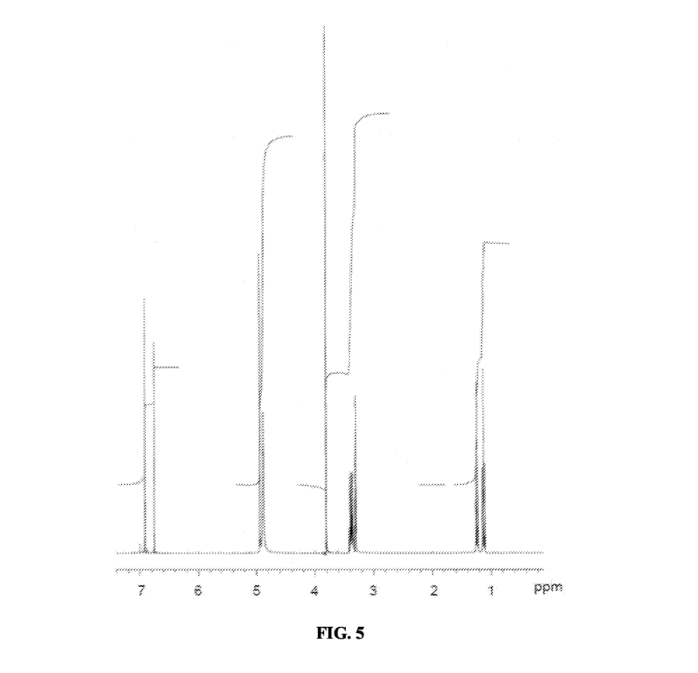

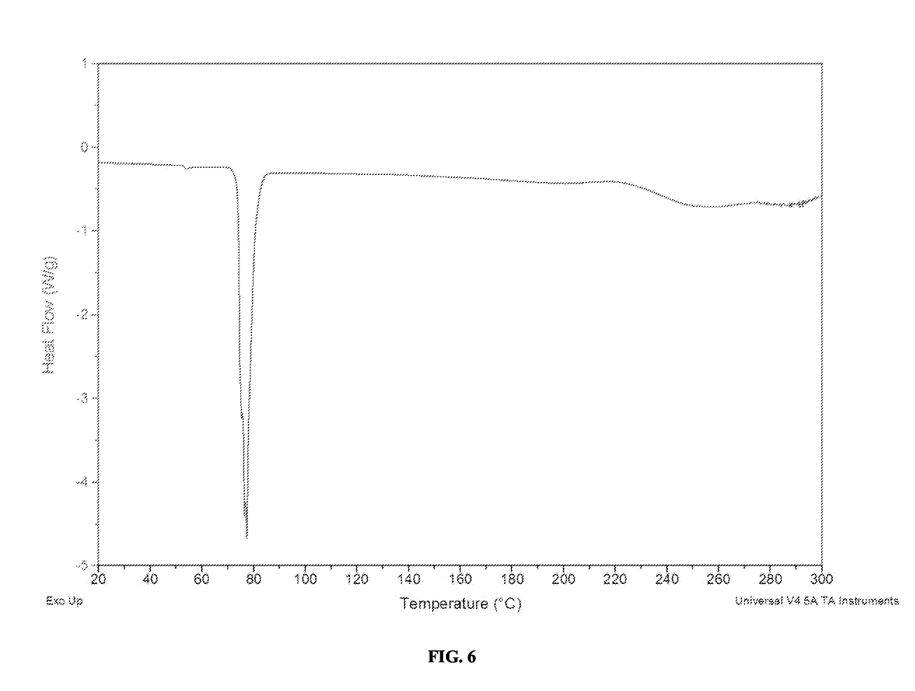

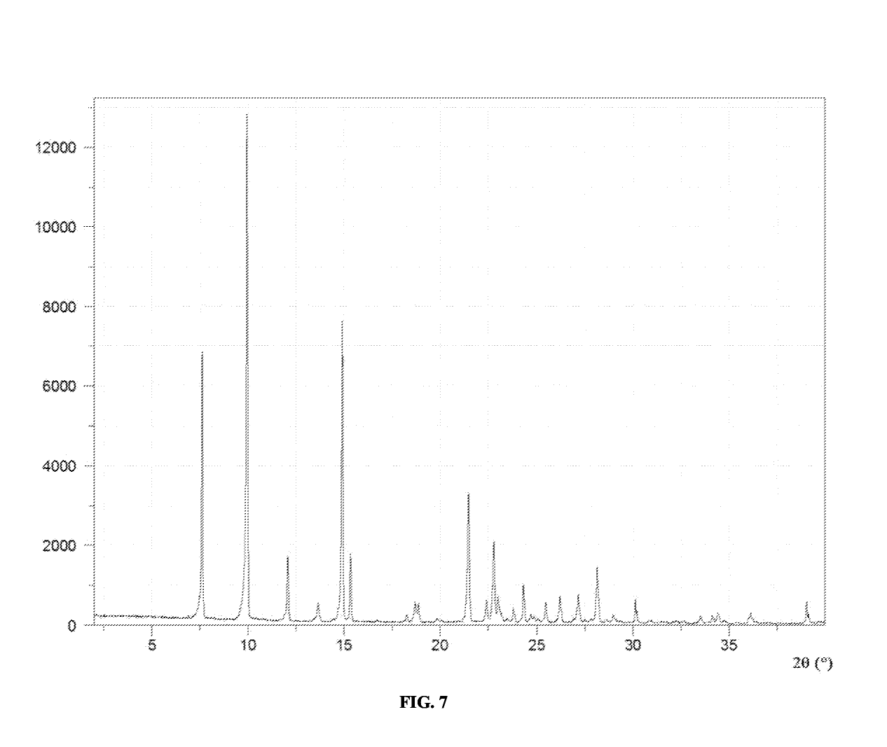

FIG. 4 is an X-ray powder diffractogram showing the diffraction pattern measured using Cu-K_{α} radiation of the (N,N-Diethylcarbamoyl)methyl methyl (2E)but-2-ene-1,4-dioate:fumaric acid cocrystal. Table 3 lists the approximate numerical values of the XRPD peak positions of the FIG. 4 diffractogram. While the entire diffractogram of FIG. 4 can be used to characterize the cocrystal, the cocrystal can also be accurately characterized with a subset of that data. For example, the peak at about 9.8 °2θ in FIG. 4 is more than 0.4 °2θ away from any peak in the X-ray powder diffraction pattern of (N,N-Diethylcarbamoyl)methyl methyl (2E)but-2-ene-1,4-dioate. In addition, the X-ray powder diffraction pattern of fumaric acid shows that no fumaric acid peak occurs within 0.4 °2θ of 9.8 °2θ. Thus, the XRPD peak at 9.8 °2θ characterizes the (N,N-Diethylcarbamoyl)methyl methyl (2E)but-2-ene-1,4-dioate:fumaric acid cocrystal.

TABLE 3
XRPD Peaks for (N,N-Diethylcarbamoyl)methyl methyl
(2E)but-2-ene-1,4-dioate:fumaric acid cocrystal

| | | | | |
|---|---|---|---|---|
| Pos. | | | | |
| [°2Th.] | Height [cts] | FWHM [°2Th.] | d-spacing [Å] | Rel. Int. [%] |

Peaks (More Characteristic)

| | | | | |
|---|---|---|---|---|
| 9.8 | 6257.2 | 0.1 | 9.0 | 100.0 |
| 21.5 | 2936.9 | 0.1 | 4.1 | 46.9 |
| 15.1 | 2412.0 | 0.1 | 5.9 | 38.6 |
| 28.3 | 1464.1 | 0.1 | 3.2 | 23.4 |
| 22.5 | 1349.7 | 0.1 | 4.0 | 21.6 |

Peaks (All)

| | | | | |
|---|---|---|---|---|
| 7.8 | 523.1 | 0.1 | 11.3 | 8.4 |
| 9.8 | 6257.2 | 0.1 | 9.0 | 100.0 |
| 12.2 | 544.2 | 0.1 | 7.3 | 8.7 |
| 13.5 | 85.2 | 0.1 | 6.5 | 1.4 |
| 13.9 | 117.8 | 0.1 | 6.4 | 1.9 |
| 15.1 | 2412.0 | 0.1 | 5.9 | 38.6 |
| 15.7 | 69.4 | 0.1 | 5.7 | 1.0 |
| 16.9 | 9.4 | 0.4 | 5.2 | 0.2 |
| 18.4 | 69.1 | 0.1 | 4.8 | 1.1 |
| 19.0 | 169.7 | 0.1 | 4.7 | 2.7 |
| 19.2 | 156.4 | 0.1 | 4.6 | 2.5 |
| 20.1 | 93.7 | 0.1 | 4.4 | 1.5 |
| 21.5 | 2935.9 | 0.1 | 4.1 | 46.9 |
| 22.5 | 1349.7 | 0.1 | 4.0 | 21.6 |
| 22.7 | 319.5 | 0.1 | 3.9 | 5.1 |
| 23.2 | 417.0 | 0.1 | 3.8 | 6.7 |
| 24.1 | 129.6 | 0.1 | 3.7 | 2.1 |
| 24.5 | 362.7 | 0.1 | 3.6 | 5.8 |
| 24.8 | 101.5 | 0.1 | 3.6 | 1.6 |
| 25.1 | 89.8 | 0.1 | 3.6 | 1.4 |
| 25.4 | 596.0 | 0.1 | 3.5 | 9.5 |
| 26.5 | 514.9 | 0.1 | 3.4 | 8.2 |
| 27.7 | 214.3 | 0.1 | 3.2 | 3.4 |
| 28.3 | 1464.1 | 0.1 | 3.2 | 23.4 |
| 28.8 | 67.2 | 0.1 | 3.1 | 1.1 |
| 29.2 | 265.8 | 0.1 | 3.1 | 4.3 |
| 30.5 | 116.3 | 0.1 | 2.9 | 1.9 |
| 31.8 | 28.6 | 0.1 | 2.8 | 0.5 |
| 32.2 | 37.4 | 0.1 | 2.8 | 0.6 |
| 34.1 | 194.8 | 0.1 | 2.6 | 3.1 |
| 34.8 | 65.5 | 0.2 | 2.6 | 1.1 |
| 36.0 | 168.0 | 0.1 | 2.5 | 2.7 |
| 39.3 | 40.0 | 0.1 | 2.3 | 0.6 |

In another example, the XRPD peak at about 15.1 °2θ in FIG. 4 is more than 0.4 °2θ away from any peak in the X-ray powder diffraction pattern of (N,N-Diethylcarbamoyl)methyl methyl (2E)but-2-ene-1,4-dioate. In addition, the X-ray diffraction pattern of fumaric acid shows that no fumaric acid peak occurs within 0.4 °2θ of 15.1 °2θ. Thus, the peak at 15.1 °2θ is another peak that alone or together with the peak at 9.3 °2θ characterizes the (N,N-Diethylcarbamoyl)methyl methyl (2E)but-2-ene-1,4-dioate:fumaric acid cocrystal.

Likewise, no XRPD peak of (N,N-Diethylcarbamoyl)methyl methyl (2E)but-2-ene-1,4-dioate or the fumaric acid diffraction pattern appears within 0.4 °2θ of 28.3 °2θ in FIG. 4, which is a peak in the (N,N-Diethylcarbamoyl)methyl methyl (2E)but-2-ene-1,4-dioate:fumaric acid cocrystal. Thus, the peak at 28.3 °2θ is another peak that alone or together with the peaks at 9.8 °2θ and/or 15.1 °2θ characterizes the (N,N-Diethylcarbamoyl)methyl methyl (2E)but-2-ene-1,4-dioate:fumaric acid cocrystal.

Also, no XRPD peak of (N,N-Diethylcarbamoyl)methyl methyl (2E)but-2-ene-1,4-dioate or the fumaric acid diffraction pattern appears within 0.4 °2θ of 25.4 °2θ in FIG. 4, which is a peak in the (N,N-Diethylcarbamoyl)methyl methyl (2E)but-2-ene-1,4-dioate:fumaric acid cocrystal. Thus, the peak at 25.4 °2θ is another peak that alone or together with the peaks at 9.8 °2θ, 15.1 °2θ, and/or 28.3 °2θ characterizes the (N,N-Diethylcarbamoyl)methyl methyl (2E)but-2-ene-1,4-dioate:fumaric acid cocrystal.

In certain embodiments, the (N,N-Diethylcarbamoyl)methyl methyl (2E)but-2-ene-1,4-dioate fumaric acid cocrystal exhibits characteristic scattering angles (2θ) at least at 9.8±0.2°, 15.1±0.2°, 21.5±0.2°, 22.5±0.2°, and 28.3±0.2° in an X-ray powder diffraction pattern measured using Cu-K_{α} radiation.

In certain embodiments, the (N,N-Diethylcarbamoyl)methyl methyl (2E)but-2-ene-1,4-dioate:fumaruc acid cocrystal exhibits characteristic scattering angles (2θ) at least a 7.8±0.2°, 9.8±0.2°, 12.2±0.2°, 15.1±0.2°, 21.5±0.2°, 22.5±0.2°, 22.7±0.2°, 23.2±0.2°, 24.5±0.2°, 25.4±0.2°, 26.5±0.2°, 27.7±0.2°, 28.3±0.2°, 29.2±0.2°, and 34.1±0.2° in an X-ray powder diffraction pattern measured using Cu-K_{α} radiation.

In certain embodiments, the (N,N-Diethylcarbamoyl)methyl methyl (2E)but-2-ene-1,4-dioate:fumaric acid cocrystal exhibits characteristic scattering angles (2θ) at least at 7.8±0.2°, 9.8±0.2°, 12.2±0.2°, 15.1±0.2°, 21.5±0.2°, 22.5±0.2°, 22.7±0.2°, 23.2±0.2°, 24.5±0.2°, 25.4±0.2°, 26.5±0.2°, 27.7±0.2°, 28.3±0.2°, 29.2±0.2°, and 34.1±0.2° in an X-ray powder diffraction pattern measured using Cu-K_{α} radiation.

In certain embodiments, the (N,N-Diethylcarbamoyl)methyl methyl (2E)but-2-ene-1,4-dioate:fumaric acid cocrystal exhibits characteristic scattering angles (2θ) at least at 9.8±0.1°, 15.1±0.1°, 21.5±0.1°, 22.5±0.1°, and 28.3±0.1° in an X-ray powder diffraction pattern measured using Cu-K_{α} radiation.

In certain embodiments, the (N,N-Diethylcarbamoyl)methyl methyl (2E)but-2-ene-1,4-dioate:fumaric acid cocrystal exhibits characteristic scattering angles (2θ) at least at 7.8±0.1°, 9.8±0.1°, 12.2±0.1°, 15.1±0.1°, 21.5±0.1°, 22.5±0.1°, 23.2±0.1°, 25.4±0.1°, 26.5±0.1° and 28.3±0.1° in an X-ray powder diffraction pattern measured using Cu-K_{α} radiation.

In certain embodiments, the (N,N-Diethylcarbamoyl)methyl methyl (2E)but-2-ene-1,4-dioate:fumaric acid cocrystal exhibits characteristic scattering angles (2θ) at least at 7.8±0.1°, 9.8±0.1°, 12.2±0.1°, 15.1±0.1°, 21.5±0.1°, 22.5 ±0.1°, 22.7±0.1°, 23.2±0.1°, 24.5±0.1°, 25.4±0.1°, 26.5±0.1°, 27.7±0.1°, 28.3±0.1°, 29.2±0.1°, and 34.1±0.1° in an X-ray powder diffraction pattern measured using Cu-K_{α} radiation.

FIG. 5 is a spectrogram showing the NMR spectrum of the (N,N-Diethylcarbamoyl)methyl methyl (2E)but-2-ene-1,4-dioate:fumaric acid cocrystal. The NMR spectral pattern indicates ¹H NMR (MeOH-d₃, 400 MHz): δ 6.99-6.90 (m, 2H), 6.75 (s, 1H), 4.93 (s, 2H), 3.80 (s, 3H), 3.39 (m, Hz, 4H), 1.24 (t, J=7.2 Hz, 3H), 1.12 (t, J=7.2 Hz, 3H) tor the (N,N-Diethylcarbamoyl)methyl methyl (2E)but-2-ene-1,4-dioate:fumaric acid cocrystal.

FIG. 6 is a DSC thermogram of the (N,N-Diethylcarbamoyl)methyl methyl (2E)but-2-ene-1,4-dioate:fumaric acid cocrystal. The thermogram shows the cocrystal has a melting point of about 74° C.

The (N,N-Diethylcarbamoyl)methyl methyl (2E)but-2-ene-1,4-dioate:fumaric acid cocrystal is expected to have a good toxicology profile, since fumaric acid is known to

be safe and appears on the GRAS list from the U.S. Food and Drug Administration.

(N,N-Diethylcarbamoyl)methyl methyl (2E)but-2-ene-1,4-dioate:Succinic Acid Cocrystal

Another cocrystal disclosed herein is a cocrystal of (N,N-Diethylcarbamoyl)methyl methyl (2E)but-2-ene-1,4-dioate and succinic acid. HPLC data indicates that the stoichiometry of (N,N-Diethylcarbamoyl)methyl methyl (2E)but-2-ene-1,4-dioate to succinic acid is 2.1. Differential scanning calorimetry (DSC) analysis of this cocrystal shows a melting point between about 62° C. and about 66° C., in certain embodiments between about 63° C. and about 65° C., and in certain embodiments at about 64° C.

FIG. 7 is an X-ray powder diffractogram showing the diffraction pattern measured using Cu-K_{α} radiation of the (N,N-Diethylcarbamoyl)methyl methyl (2E)but-2-ene-1,4-dioate:succinic acid cocrystal. Table 4 lists the approximate numerical values of the XRPD peak positions of the FIG. 7 diffractogram. While the entire diffractogram of FIG. 7 can be used to characterize the cocrystal, the cocrystal can also be accurately characterized with a subset of that data. For example, the XRPD peak at about 10.0 °2θ in FIG. 7 is more than 0.4 °2θ away from any XRPD peak of (N,N-Diethylcarbamoyl)methyl methyl (2E)but-2-ene-1,4-dioate. In addition, the X-ray diffraction pattern of succinic acid shows that no succinic acid XRPD peak occurs within 0.4 °2θ of 10.0 °2θ. Thus, the XRPD peak at 10.0 °2θ characterizes the (N,N-Diethylcarbamoyl)methyl methyl (2E)but-2-ene-1,4-dioate:succinic acid cocrystal.

TABLE 4
XRPD Peaks for (N,N-Diethylcarbamoyl)methyl methyl
(2E)but-2-ene-1,4-dioate:succinic acid cocrystal

| | | | | |
|---|---|---|---|---|
| Pos. | | | | |
| [°2Th.] | Height [cts] | FWHM [°2Th.] | d-spacing [Å] | Rel. Int. [%] |

Peaks (More Characteristic)

| | | | | |
|---|---|---|---|---|
| 10.0 | 12620.0 | 0.1 | 8.9 | 100.0 |
| 14.9 | 7374.7 | 0.1 | 5.9 | 58.4 |
| 7.6 | 6639.6 | 0.1 | 11.6 | 52.6 |
| 21.5 | 3253.7 | 0.1 | 4.1 | 25.8 |
| 22.8 | 1981.8 | 0.1 | 3.9 | 15.7 |

Peaks (All)

| | | | | |
|---|---|---|---|---|
| 7.6 | 6639.6 | 0.1 | 11.6 | 52.6 |
| 10.0 | 12620.0 | 0.1 | 8.9 | 100.0 |
| 12.1 | 1584.1 | 0.1 | 7.3 | 12.6 |
| 13.6 | 448.5 | 0.1 | 6.5 | 3.6 |
| 14.9 | 7374.7 | 0.1 | 5.9 | 58.4 |
| 15.3 | 1677.9 | 0.1 | 5.8 | 13.3 |
| 18.2 | 188.8 | 0.1 | 4.9 | 1.5 |
| 18.7 | 513.4 | 0.1 | 4.7 | 4.1 |
| 18.9 | 448.8 | 0.1 | 4.7 | 3.6 |
| 19.8 | 85.7 | 0.1 | 4.5 | 0.7 |
| 21.5 | 3253.7 | 0.1 | 4.1 | 25.8 |
| 22.4 | 548.6 | 0.1 | 4.0 | 4.4 |
| 22.8 | 1981.8 | 0.1 | 3.9 | 15.7 |
| 23.0 | 657.8 | 0.1 | 3.9 | 5.2 |
| 23.5 | 101.2 | 0.1 | 3.8 | 0.8 |
| 23.8 | 358.9 | 0.1 | 3.7 | 2.8 |
| 24.3 | 908.2 | 0.1 | 3.7 | 7.2 |
| 24.7 | 202.1 | 0.1 | 3.6 | 1.6 |
| 24.9 | 170.9 | 0.1 | 3.6 | 1.4 |
| 25.5 | 494.7 | 0.1 | 3.5 | 3.9 |
| 26.2 | 637.6 | 0.1 | 3.4 | 5.1 |
| 77.2 | 703.9 | 0.1 | 3.3 | 5.6 |
| 27.5 | 71.7 | 0.1 | 3.2 | 0.6 |
| 28.1 | 1337.4 | 0.1 | 3.2 | 10.6 |
| 28.6 | 76.1 | 0.1 | 3.1 | 0.6 |
| 29.0 | 214.1 | 0.1 | 3.1 | 1.7 |
| 30.1 | 545.0 | 0.1 | 3.0 | 4.3 |
| 30.9 | 62.5 | 0.2 | 2.9 | 0.5 |
| 31.6 | 19.4 | 0.3 | 2.8 | 0.2 |
| 32.2 | 53.6 | 0.2 | 2.8 | 0.4 |
| 32.6 | 65.3 | 0.1 | 2.7 | 0.5 |
| 33.5 | 163.4 | 0.1 | 2.7 | 1.3 |
| 34.1 | 182.1 | 0.1 | 2.6 | 1.4 |
| 34.4 | 251.6 | 0.1 | 2.6 | 2.0 |
| 34.7 | 61.6 | 0.2 | 2.6 | 0.5 |
| 36.1 | 264.6 | 0.1 | 2.5 | 2.1 |
| 39.0 | 504.3 | 0.1 | 2.3 | 4.0 |
| 39.1 | 202.0 | 0.1 | 2.3 | 1.6 |

In another example, the XRPD peak at about 14.9 °2θ in FIG. 7 is more than 0.4 °2θ away from any XRPD peak of (N,N-Diethylcarbamoyl)methyl methyl (2E)but-2-ene-1,4-dioate. In addition, the X-ray diffraction pattern of succinic acid shows that no succinic acid XRPD peak occurs within 0.4 °2θ of 14.9 °2θ. Thus, the XRPD peak at 14.9 °2θ is another peak that alone or together with the peak at 10.0 °2θ characterizes the (N,N-Diethylcarbamoyl)methyl methyl (2E)but-2-ene-1,4-dioate:succinic acid cocrystal.

Likewise, no XRPD peak of (N,N-Diethylcarbamoyl)methyl methyl (2E)but-2-ene-1,4-dioate or the succinic acid diffraction patterns appear within 0.4 °2θ of 7.6 °2θ in FIG. 7, which is a peak in the (N,N-Diethylcarbamoyl)methyl methyl (2E)but-2-ene-1,4-dioate:succinic acid cocrystal. Thus, the peak at 7.6 °2θ is another peak that alone or together with the peaks at 10.0 °2θ and/or 14.9 °2θ characterizes the (N,N-Diethylcarbamoyl)methyl methyl (2E)but-2-ene-1,4-dioate:succinic acid cocrystal.

Also, no XRPD peak of (N,N-Diethylcarbamoyl)methyl methyl (2E)but-2-ene-1,4-dioate or the succinic acid diffraction pattern appear within 0.4 °2θ of 15.3 °2θ in FIG. 7, which is a peak in the (N,N-Diethylcarbamoyl)methyl methyl (2E)but-2-ene-1,4-dioate:succinic acid cocrystal. Thus, the peak at 15.3 °2θ is another peak that alone or together with the peaks at 10.0 °2θ, 14.9 °2θ and/or 7.6 °2θ characterizes the (N,N-Diethylcarbamoyl)methyl methyl (2E)but-2-ene-1,4-dioate:succinic acid cocrystal.

Also, no XRPD peak of (N,N-Diethylcarbamoyl)methyl methyl (2E)but-2-ene-1,4-dioate or the succinic acid diffraction pattern appears within 0.4 °2θ of 28.1 °2θ in FIG. 7, which is a peak in the (N,N-Diethylcarbamoyl)methyl methyl (2E)but-2-ene-1,4-dioate:succinic acid cocrystal. Thus, the peak at 28.1 °2θ is another peak that alone or together with the peaks at 10.0 °2θ, 14.9 °2θ, 7.6 °2θ and/or 15.3 °2θ characterizes the (N,N-Diethylcarbamoyl)methyl methyl (2E)but-2-ene-1,4-dioate:succinic acid cocrystal.

In certain embodiments, the (N,N-Diethylcarbamoyl)methyl methyl (2E)but-2-ene-1,4-dioate:succinic acid cocrystal exhibits characteristic scattering angles (2θ) at least at 7.6±0.2°, 10.0±0.2°, 14.9±0.2°, 21.5±0.2° and 22.8±0.2° in an X-ray powder diffraction pattern measured using Cu-K_{α} radiation.

in certain embodiments, the (N,N-Diethylcarbamoyl)methyl methyl (2E)but-2-ene-1,4-dioate:succinic acid cocrystal exhibits characteristic scattering angles (2θ) at least at 7.8±0.2°, 10.0±0.2°, 12.1±0 2°, 14.9±0.2 , 15.3±0.2°, 21.5±0.2°, 22.8±0.2°, 24.3±0.2°, 27.2±0.2° and 28.1±0.2° in an X-ray powder diffraction pattern measured using Cu-K_{α} radiation.

In certain embodiments, the (N,N-Diethylcarbamoyl)methyl methyl (2E)but-2-ene-1,4-dioate:succinic acid cocrystal exhibits characteristic scattering angles (2θ) at least at 7.6±0.2°, 10.0±0.2°, 12.1±0.2°, 14.9±0.2°, 15.3±0.2°, 18.7±0.2°, 21.5±0.2°, 22.4±0.2°, 22.8±0.2°, 23.0±0.2°, 24.3±0.2°, 26.2±0.2°, 27.2±0.2°, 28.1±0.2° and 30.1±0.2° in an X-ray powder diffraction pattern measured using Cu-K_{α} radiation.

In certain embodiments, the (N,N-Diethylcarbamoyl)methyl methyl (2E)but-2-ene-1,4-dioate:succinic acid cocrystal exhibits characteristic scattering angles (2θ) at least at 7.8±0.1°, 10.0±0.1°, 14.9±0.1°, 21.5±0.1° and 22.8±0.1° in an X-ray powder diffraction pattern measured using Cu-K_{α} radiation.

In certain embodiments, the (N,N-Diethylcarbamoyl)methyl methyl (2E)but-2-ene-1,4-dioate:succinic acid cocrystal exhibits characteristic scattering angles (2θ) at least at 7.6±0.1°, 10.0±0.1°, 12.1±0.1°, 14.9±0.1°, 15.3±0.1°, 21.5±0.1°, 22.8±0.1°, 24.3±0.1°, 27.2±0.1° and 28.1±0.1° in an X-ray powder diffraction pattern measured using Cu-K_{α} radiation.

In certain embodiments, the (N,N-Diethylcarbamoyl)methyl methyl (2E)but-2-ene-1,4-dioate:succinic acid cocrystal exhibits characteristic scattering angles (2θ) at least at 7.6±0.1°, 10.0±0.1°, 12.1±0.1°, 14.9±0.1°, 15.3±0.1°, 13.7±0.1°, 21.5±0.1°, 22.4±0.1°, 22.8±0.1°, 23.0±0.1°, 24.3±0.1°, 26.2±0.1°, 27,2±0.1°, 28.1±0.1° and 30.1±0.1° in an X-ray powder diffraction pattern measured using Cu-K_{α} radiation.

FIG. 8 is a spectrogram showing the NMR spectrum of the (N,N-Diethylcarbamoyl)methyl methyl (2E)but-2-ene-1,4-dioate:succinic acid cocrystal. The NMR spectral pattern indicates ¹H NMR (MeOH-d₃, 400 MHz); δ 6.99-6.90 (m, 2H), 4.93 (s, 2H), 3.80 (s, 3H), 3.39 (m, Hz, 4H), 2.55 (s, 2H), 1.24 (t, J=7.2 Hz, 3H), 1.12 (t, J=7.2 Hz, 3H) for the (N,N-Diethylcarbamoyl)methyl methyl (2E)but-2-ene-1,4-dioate:succinic acid cocrystal.

FIG. 9 is a DSC thermogram of the (N,N-Diethylcarbamoyl)methyl methyl (2E)but-2-ene-1,4-dioate:succinic acid cocrystal. The thermogram shows the cocrystal has a melting point of about 64° C.

The (N,N-Diethylcarbamoyl)methyl methyl (2E)but-2-ene-1,4-dioate:succinic acid cocrystal is expected to have a good toxicology profile, since succinic acid is known to be safe and appears on the GRAS list from the U.S. Food and Drug Administration.

(N,N-Diethylcarbamoyl)methyl methyl (2E)but-2-ene-1,4-dioate:Maleic Acid Cocrystal

Another cocrystal disclosed herein is a cocrystal of (N,N-Diethylcarbamoyl)methyl methyl (2E)but-2-ene-1,4-dioate and maleic acid. HPLC data indicates that the stoichiometry of (N,N-Diethylcarbamoyl)methyl methyl (2E)but-2-ene-1,4-dioate to maleic acid is 1:1. Differential scanning calorimetry (DSC) analysis of this cocrystal shows a melting point between about 65° C. and about 69° C., in some embodiments between about 66° C. and about 68° C., and in certain embodiments at about 67° C.

FIG. 10 is an X-ray powder diffractogram showing the diffraction pattern measured using Cu-K_{α} radiation of the (N,N-Diethylcarbamoyl)methyl methyl (2E)but-2-ene-1,4-dioate:maleic acid cocrystal. Table 5 lists the approximate numerical values of the XRPD peak positions of the FIG. 10 diffractogram. While the entire diffractogram of FIG. 10 can be used to characterize the cocrystal the cocrystal can also be accurately characterized with a subset of that data. For example, the peak at about 7.7 °2θ in FIG. 10 is more than 0.4 °2θ away from any peak in the X-ray powder diffraction pattern of (N,N-Diethylcarbamoyl)methyl methyl (2E)but-2-ene-1,4-dioate. In addition, the X-ray diffraction pattern of maleic acid shows that no maleic acid XRPD peak occurs within 0.4 °2θ of 7.7 °2θ. Thus, the XRPD peak at 7.7 °2θ characterizes the (N,N-Diethylcarbamoyl)methyl methyl (2E)but-2-ene-1,4-dioate:maleic acid cocrystal.

TABLE 5
XRPD Peaks for (N,N-Diethylcarbamoyl)methyl methyl
(2E)but-2-ene-1,4-dioate:maleic acid cocrystal

| | | | | |
|---|---|---|---|---|
| Pos. | | | | |
| [°2Th.] | Height [cts] | FWHM [°2Th.] | d-spacing [Å] | Rel. Int. [%] |

Peaks (More Characteristic)

| | | | | |
|---|---|---|---|---|
| 7.7 | 12293.9 | 0.1 | 11.5 | 100.0 |
| 7.4 | 5135.7 | 0.1 | 11.9 | 41.8 |
| 10.0 | 3416.6 | 0.1 | 8.9 | 27.8 |
| 20.0 | 2062.3 | 0.1 | 4.4 | 16.8 |
| 13.2 | 2004.9 | 0.1 | 6.7 | 16.3 |

Peaks (All)

| | | | | |
|---|---|---|---|---|
| 7.4 | 5135.7 | 0.1 | 11.9 | 41.8 |
| 7.7 | 12293.9 | 0.1 | 11.5 | 100.0 |
| 10.0 | 3416.6 | 0.1 | 8.9 | 27.8 |
| 12.7 | 654.7 | 0.1 | 7.0 | 5.3 |
| 13.2 | 2004.9 | 0.1 | 6.7 | 16.3 |
| 14.9 | 645.8 | 0.1 | 6.0 | 5.3 |
| 16.3 | 145.3 | 0.1 | 5.4 | 1.2 |
| 16.7 | 1227.9 | 0.1 | 5.3 | 10.0 |
| 17.2 | 516.8 | 0.1 | 5.2 | 4.2 |
| 17.6 | 96.9 | 0.1 | 5.0 | 0.8 |
| 18.2 | 270.4 | 0.1 | 4.9 | 2.2 |
| 18.6 | 229.7 | 0.1 | 4.8 | 1.9 |
| 19.6 | 539.4 | 0.1 | 4.5 | 4.4 |
| 20.0 | 2062.3 | 0.1 | 4.4 | 16.8 |
| 20.3 | 180.7 | 0.1 | 4.4 | 1.5 |
| 20.6 | 457.1 | 0.1 | 4.3 | 3.7 |
| 21.6 | 205.0 | 0.1 | 4.1 | 1.7 |
| 22.4 | 498.9 | 0.1 | 4.0 | 4.1 |
| 22.6 | 410.4 | 0.1 | 3.9 | 3.3 |
| 23.2 | 1263.9 | 0.1 | 3.8 | 10.3 |
| 24.1 | 741.8 | 0.1 | 3.7 | 6.0 |
| 24.6 | 1862.9 | 0.1 | 3.6 | 15.2 |
| 25.1 | 104.1 | 0.2 | 3.5 | 0.9 |
| 25.9 | 363.0 | 0.1 | 3.4 | 3.0 |
| 26.7 | 270.7 | 0.2 | 3.3 | 2.2 |
| 27.3 | 1033.6 | 0.1 | 3.3 | 8.4 |
| 27.6 | 212.7 | 0.1 | 3.2 | 1.7 |
| 27.9 | 208.3 | 0.1 | 3.2 | 1.7 |
| 28.1 | 760.5 | 0.1 | 3.2 | 6.2 |
| 29.7 | 206.2 | 0.2 | 3.0 | 1.7 |
| 30.1 | 935.2 | 0.2 | 3.0 | 7.6 |
| 31.6 | 135.0 | 0.1 | 2.8 | 1.1 |
| 31.8 | 125.6 | 0.1 | 2.8 | 1.0 |
| 32.3 | 113.4 | 0.2 | 2.8 | 0.9 |
| 34.8 | 240.8 | 0.1 | 2.6 | 2.0 |
| 37.5 | 96.6 | 0.1 | 2.4 | 0.8 |

In another example, the XRPD peak at about 7.4 °2θ in FIG. 10 is more than 0.4 °2θ away from any peak in the X-ray powder diffraction pattern of (N,N-Diethylcarbamoyl)methyl methyl (2E)but-2-ene-1,4-dioate. In addition, the X-ray diffraction pattern of maleic acid shows that no maleic acid peak occurs within 0.4 °2θ of 7.4 °2θ. Thus, the XRPD peak at 7.4 °2θ is another peak that alone or together with the peak at 7.7 °2θ characterizes the (N,N-Diethylcarbamoyl)methyl methyl (2E)but-2-ene-1,4-dioate:maleic acid cocrystal.

Likewise, no XRPD peak of (N,N-Diethylcarbamoyl)methyl methyl (2E)but-2-ene-1,4-dioate or the maleic acid diffraction pattern appears within 0.4 °2θ of 10.0 °2θ in FIG. 10, which is a peak in the (N,N-Diethylcarbamoyl)methyl methyl (2E)but-2-ene-1,4-dioate:maleic acid cocrystal. Thus, the peak at 10.0 °2θ is another peak that alone or together with the peaks at 7.7 °2θ and/or 7.4 °2θ characterizes the (N,N-Diethylcarbamoyl)methyl methyl (2E)but-2-ene-1,4-dioate:maleic acid cocrystal.

Likewise, no XRPD peak of (N,N-Diethylcarbamoyl)methyl methyl (2E)but-2-ene-1,4-dioate or the maleic acid diffraction pattern appears within 0.4 °2θ of 13.2 °2θ, which is a peak in the (N,N-Diethylcarbamoyl)methyl methyl (2E)but-2-ene-1,4-dioate:maleic acid cocrystal. Thus, the peak at 13.2 °2θ is another peak that alone or together with the peaks at 7.7 °2θ, 7.4 °2θ and/or 10.0 °2θ characterizes the (N,N-Diethylcarbamoyl)methyl methyl (2E)but-2-ene-1,4-dioate:maleic acid cocrystal.

Likewise, no XRPD peak of (N,N-Diethylcarbamoyl)methyl methyl (2E)but-2-ene-1,4-dioate or the maleic acid diffraction pattern appears within 0.4°2θ of 30.1°2θ in FIG. 10. which is a peak in the (N,N-Diethylcarbamoyl)methyl methyl (2E)but-2-ene-1,4-dioate:maleic acid cocrystal. Thus, the peak at 30.1 °2θ is another peak that alone or together with the peaks at 7.7 °2θ, 7.4 °2θ, 10.0 °2θ and/or 13.2 °2θ characterizes the (N,N-Diethylcarbamoyl)methyl methyl (2E)but-2-ene-1,4-dioate: maleic acid cocrystal.

In certain embodiments, the (N,N-Diethylcarbamoyl)methyl methyl (2E)but-2-ene-1,4-dioate:maleic acid cocrystal exhibits characteristic scattering angles (2θ) at least at 7.4±0.2°, 7.7±0.2°, 10.0±0.2°, 13.2±0.2° and 20.0±0.2° in an X-ray powder diffraction pattern measured using Cu-K_{α} radiation.

In certain embodiments, the (N,N-Diethylcarbamoyl)methyl methyl (2E)but-2-ene-1,4-dioate:maleic acid cocrystal exhibits characteristic scattering angles (2θ) at least at 7.4±0.2°, 7.7±0.2°, 10.0±0.2°, 13.2±0.2°, 16.7±0.2°, 20.0±0.2°, 23.2±0.2°, 24.6±0.2°, 27.3±0.2° and 30.1±0.2° in an X-ray powder diffraction pattern measured using Cu-K_{α} radiation.

In certain embodiments, the (N,N-Diethylcarbamoyl)methyl methyl (2E)but-2-ene-1,4-dioate:maleic acid cocrystal exhibits characteristic scattering angles (2θ) at least at 7.4±0.2°, 7.7±0.2°, 10.0±0 2°, 12.7±0.2°, 13.2±0.2°, 14.9±0.2°, 16.7±0.2°, 19.6±0.2°, 20.0±0.2°, 23.2±0.2°, 24.1±0.2°, 24.6±0.2°, 27.3±0.2°, 28.1±0.2° and 30.1±0.2° in an X-ray powder diffraction pattern measured using Cu-_{α} radiation.

In certain embodiments, the (N,N-Diethylcarbamoyl)methyl methyl (2E)but-2-ene-1,4-dioate: maleic acid cocrystal exhibits characteristic scattering angles (2θ) at least at 7.4±0.1°, 7.7±0.1°, 10.0±0.1°, 13.2±0.1° and 20.0±0.1° in an X-ray powder diffraction pattern measured using Cu-K_{α} radiation.

in certain embodiments, the (N,N-Diethylcarbamoyl)methyl methyl (2E)but-2-ene-1,4-dioate:maleic acid cocrystal exhibits characteristic scattering angles (2θ) at least at 7.4±0.1°, 7.7±0.1°, 10.0±0.1°, 13.2±0.1°, 16.7±0.1°, 20.0±0.1°, 23.2±0.1°, 24.6±0.1°, 27.3±0.1° and 30.1±0.1° in an X-ray powder diffraction pattern measured using Cu-K_{α} radiation.

In certain embodiments, the (N,N-Diethylcarbamoyl)methyl methyl (2E)but-2-ene-1,4-dioate:maleic acid cocrystal exhibits characteristic scattering angles (2θ) at least at 7.4±0.1°, 7.7±0.1°, 10.0±0.1°, 12.7±0.1°, 13.2±0.1°, 14.9±0.1°, 16.7±0.1°, 19.6±0.1°, 20.0±0.1°, 23.2±0.1°, 24.1±0.1°, 24.6±0.1°, 27.3±0.1°, 28.1±0.1° and 30.1±0.1° in an X-ray powder diffraction pattern measured using Cu-K_{α} radiation.

FIG. 11 is a spectrogram showing the NMR spectrum of the (N,N-Diethylcarbamoyl)methyl methyl (2E)but-2-ene-1,4-dioate:maleic acid cocrystal. The NMR spectral pattern indicates ¹¹H NMR (CDCl₃, 400 MHz): δ 6.99-6.90 (m, 2H), 6.38 (s, 2H), 4.83 (s, 2H), 3.80 (s, 3H), 3.39 (q, J=7.2 Hz, 2H), 3.26 (q, J=7.2 Hz, 2H), 1.24 (t, J=7.2 Hz, 3H), 1.14 (t, J=7.2 Hz, 3H) for the (N,N-Diethylcarbamoyl)methyl methyl (2E)but-2-ene-1,4-dioate:maleic acid cocrystal.

FIG. 12 is a DSC thermogram of the (N,N-Diethylcarbamoyl)methyl methyl (2E)but-2-ene-1,4-dioate:maleic acid cocrystal. The thermogram shows the cocrystal has a melting point of about 67° C.

The (N,N-Diethylcarbamoyl)methyl methyl (2E)but-2-ene-1,4-dioate:maleic acid cocrystal is expected to have a good toxicology profile, since maleic acid is known to be safe and appears on the GRAS list from the U.S. Food and Drug Administration.

(N,N-Diethylcarbamoyl)methyl methyl (2E)but-2-ene-1,4-dioate:Malic Acid Cocrystal

Another cocrystal disclosed herein is a cocrystal of (N,N-Diethylcarbamoyl)methyl methyl (2E)but-2-ene-1,4-dioate and malic acid. HPLC data indicates that the stoichiometry of (N,N-Diethylcarbamoyl)methyl methyl (2E)but-2-ene-1,4-dioate to malic acid is 1:1. Differential scanning calorimetry (DSC) analysis of this cocrystal shows a melting point between about 61° C. and about 65° C., in certain embodiments between about 62° C. and about 64° C., and in certain embodiments at about 63° C.

FIG. 13 is an X-ray powder diffractogram showing the diffraction pattern measured using Cu-K_{α} radiation of the (N,N-Diethylcarbamoyl)methyl methyl (2E)but-2-ene-1,4-dioate:malic acid cocrystal. Table 6 lists the approximate numerical values of the XRPD peak positions of the diffractogram of FIG. 13. While the entire diffractogram of FIG. 13 can be used to characterize the cocrystal, the cocrystal can also be accurately characterized with a subset of that data. For example, the peak at about 5.8 °2θ in FIG. 13 is more than 0.4 °2θ away from any peak in the X-ray powder diffraction pattern of (N,N-Diethylcarbamoyl)methyl methyl (2E)but-2-ene-1,4-dioate. In addition, the X-ray diffraction pattern of malic acid shows that no malic acid peak occurs within 0.4 °2θ of 5.8 °2θ. Thus, the XRPD peak at 5.8 °2θ characterizes the (N,N-Diethylcarbamoyl)methyl methyl (2E)but-2-ene-1,4-dioate:malic acid cocrystal.

TABLE 6
XRPD Peaks for (N,N-Diethylcarbamoyl)methyl methyl
(2E)but-2-ene-1,4-dioate:malic acid cocrystal

| | | | | |
|---|---|---|---|---|
| Pos. | | | | |
| [°2Th.] | Height [cts] | FWHM [°2Th.] | d-spacing [Å] | Rel. Int. [%] |

Peaks (More Characteristic)

| | | | | |
|---|---|---|---|---|
| 28.3 | 2658.3 | 0.1 | 3.2 | 100.0 |
| 5.8 | 2509.4 | 0.1 | 15.1 | 94.4 |
| 22.3 | 2190.4 | 0.1 | 4.0 | 82.4 |
| 10.6 | 1978.0 | 0.1 | 8.3 | 74.4 |
| 23.9 | 1783.6 | 0.1 | 3.7 | 67.1 |

Peaks (All)

| | | | | |
|---|---|---|---|---|
| 5.8 | 2509.4 | 0.1 | 15.1 | 94.4 |
| 8.6 | 547.7 | 0.1 | 10.3 | 20.6 |
| 10.6 | 1978.0 | 0.1 | 8.3 | 74.4 |
| 11.6 | 895.0 | 0.1 | 7.6 | 33.7 |
| 14.1 | 184.7 | 0.1 | 6.3 | 7.0 |
| 14.9 | 1573.2 | 0.1 | 6.0 | 59.2 |
| 15.3 | 193.7 | 0.1 | 5.8 | 7.3 |
| 15.9 | 114.2 | 0.1 | 5.6 | 4.3 |
| 17.0 | 706.6 | 0.1 | 5.2 | 26.6 |
| 17.5 | 871.6 | 0.1 | 5.1 | 32.8 |
| 17.8 | 553.7 | 0.1 | 5.0 | 20.8 |
| 18.0 | 172.7 | 0.1 | 4.9 | 6.5 |
| 18.4 | 853.6 | 0.1 | 4.8 | 32.1 |
| 19.0 | 540.0 | 0.1 | 4.7 | 20.3 |
| 20.0 | 887.7 | 0.1 | 4.4 | 33.4 |
| 20.2 | 752.3 | 0.1 | 4.4 | 28.3 |
| 21.3 | 1473.5 | 0.1 | 4.2 | 55.4 |
| 22.3 | 2190.4 | 0.1 | 4.0 | 82.4 |
| 23.3 | 262.6 | 0.1 | 3.8 | 9.9 |
| 23.9 | 1783.6 | 0.1 | 3.7 | 67.1 |
| 25.3 | 762.5 | 0.1 | 3.5 | 28.7 |
| 25.4 | 741.9 | 0.1 | 3.5 | 27.9 |
| 25.8 | 293.6 | 0.1 | 3.5 | 11.0 |
| 26.2 | 91.7 | 0.1 | 3.4 | 3.5 |
| 26.9 | 433.3 | 0.2 | 3.3 | 16.3 |
| 27.5 | 114.7 | 0.1 | 3.2 | 4.3 |
| 28.3 | 2658.3 | 0.1 | 3.2 | 100.0 |
| 28.6 | 221.4 | 0.1 | 3.1 | 8.3 |
| 29.0 | 445.4 | 0.1 | 3.1 | 16.8 |
| 29.3 | 402.2 | 0.1 | 3.0 | 15.1 |
| 29.9 | 351.0 | 0.1 | 3.0 | 13.2 |
| 30.1 | 495.3 | 0.1 | 3.0 | 18.6 |
| 31.1 | 167.1 | 0.1 | 2.9 | 6.3 |
| 32.2 | 98.0 | 0.2 | 2.8 | 3.7 |
| 32.7 | 221.5 | 0.1 | 2.7 | 8.3 |
| 33.4 | 54.0 | 0.3 | 2.7 | 2.0 |
| 34.3 | 52.8 | 0.3 | 2.6 | 2.0 |
| 35.4 | 75.0 | 0.1 | 2.5 | 2.8 |
| 36.9 | 176.2 | 0.1 | 2.4 | 6.6 |
| 37.4 | 227.1 | 0.1 | 2.4 | 8.5 |

In another example, the XRPD peak at about 22.3 °2θ in FIG. 13 is more than 0.4 °2θ away from any peak in the X-ray powder diffraction pattern of (N,N-Diethylcarbamoyl)methyl methyl (2E)but-2-ene-1,4-dioate. In addition, the X-ray diffraction pattern of malic acid shows that no malic acid peak occurs within 0.4 °2θ of 22.3 °2θ. Thus, the XRPD peak at 22.3 °2θ is another peak that alone or together with the peak at 5.8 °2θ characterizes the (N,N-Diethylcarbamoyl)methyl methyl (2E)but-2-ene-1,4-dioate:malic acid cocrystal.

Likewise, no XRPD peak of (N,N-Diethylcarbamoyl)methyl methyl (2E)but-2-ene-1,4-dioate or the malic acid diffraction pattern appears within 0.4 °2θ of 14.9 °2θ in FIG. 13, which is a peak in the (N,N-Diethylcarbamoyl)methyl methyl (2E)but-2-ene-1,4-dioate:malic acid cocrystal. Thus, the XRPD peak at 14.9 √2θ is another peak that alone or together with the peaks at 5.8 °2θ and/or 22.3 °2θ characterizes the (N,N-Diethylcarbamoyl)methyl methyl (2E)but-2-ene-1,4-dioate:malic acid cocrystal.

Likewise, no XRPD peak of (N,N-Diethylcarbamoyl)methyl methyl (2E)but-2-ene-1,4-dioate or the malic acid diffraction pattern appears within 0.4 °2θ of 17.5 °2θ in FIG. 13, which is a peak in the (N,N-Diethylcarbamoyl)methyl methyl (2E)but-2-ene-1,4-dioate:malic acid cocrystal. Thus, the XRPD peak at 17.5 √2θ is another peak that alone or together with the peaks at 5.8 °2θ, 22.3 °2θ and/or 14.9 °2θ characterizes the (N,N-Diethylcarbamoyl)methyl methyl (2E)but-2-ene-1,4-dioate:malic acid cocrystal.

Likewise, no XRPD peak of (N,N-Diethylcarbamoyl)methyl methyl (2E)but-2-ene-1,4-dioate or the malic acid diffraction pattern appears within 0.4 °2θ of 25.3 °2θ in FIG. 13, which is a peak in the (N,N-Diethylcarbamoyl)methyl methyl (2E)but-2-ene-1,4-dioate:malic acid cocrystal. Thus, the XRPD peak at 25.3 °2θ is another peak that alone or together with the peaks at 5.8 °2θ, 22.3 °2θ, 14.9 °2θ and/or 17.5 °2θ characterizes the (N,N-Diethylcarbamoyl)methyl methyl (2E)but-2-ene-1,4-dioate:malic acid cocrystal.

In certain embodiments, the (N,N-Diethylcarbamoyl)methyl methyl (2E)but-2-ene-1,4-dioate:malic acid cocrystal exhibits characteristic scattering angles (2θ) at least at 5.8±0.2°, 10.6±0.2°, 22.3±0.2°, 23.9±0.2° and 28.3±0.2° in an X-ray powder diffraction pattern measured using Cu-K_{α} radiation.

In certain embodiments, the (N,N-Diethylcarbamoyl)methyl methyl (2E)but-2-ene-1,4-dioate;malic acid cocrystal exhibits characteristic scattering angles (2θ) at least at 5.8±0.2°, 10.6±0.2°, 11.6±0.2°, 14.9±0.2°, 17.5±0.2°, 20.0±0.2°, 21.3±0.2°, 22.3±0.2°, 23.9±0.2° and 26.3±0.2° in an X-ray powder diffraction pattern measured using Cu-K_{α} radiation.

In certain embodiments, the (N,N-Diethylcarbamoyl)methyl methyl (2E)but-2-ene-1,4-dioate:malic acid cocrystal exhibits characteristic scattering angles (2θ) at least at 5.8±0.2°, 10.6±0.2°, 11.6±0.2°, 14.9±0.2°, 17.0±0.2°, 17.5±0.2°, 18.4±0.2°, 20.0±0.2°, 20.2±0.2°, 21.3±0.2°, 22.3±0.2°, 23.9±0.2°, 25.3±0.2°, 25.4±0.2° and 28.3±0.2° in an X-ray powder diffraction pattern measured using Cu-K_{α} radiation.

In certain embodiments, the (N,N-Diethylcarbamoyl)methyl methyl (2E)but-2-ene-1,4-dioate:malic acid cocrystal exhibits characteristic scattering angles (2θ) at least at 5.8±0.1°, 10.8±0.1°, 22.3±0.1°, 23.9±0.1° and 28.3±0.1° in an X-ray powder diffraction pattern measured using Cu-K_{α} radiation.

In certain embodiments, the (N,N-Diethylcarbamoyl)methyl methyl (2E)but-2-ene-1,4-dioate:malic acid cocrystal exhibits characteristic scattering angles (2θ) at least at 5.8±0.1°, 10.6±0.1°, 11.6±0.1°, 14.9±0.1°, 17.5±0.1°, 20.0±0.1°, 21.3±0.1°, 22.3±0.1°, 23.9±0.1° and 28.3±0.1° in an X-ray powder diffraction pattern measured using Cu-K_{α} radiation.

In certain embodiments, the (N,N-Diethylcarbamoyl)methyl methyl (2E)but-2-ene-1,4-dioate:malic acid cocrystal exhibits characteristic scattering angles (2θ) at least at 5.8±0.1°, 10.6±0.1°, 11.6±0.1°, 14.9±0.1°, 17.0±0.1°, 17.5±0.1°, 18.4±0.1°, 20.0±0.1°, 20.2±0.1°, 21.3±0.1°, 22.3±0.1°, 23.9±0.1°, 25.3±0.1°, 25.4±0.1° and 28.3±0.1° in an X-ray powder diffraction pattern measured using Cu-K_{α} radiation.

FIG. 14 is a spectrogram showing the NMR spectrum of the (N,N-Diethylcarbamoyl)methyl methyl (2E)but-2-ene-1,4-dioate:malic acid cocrystal. The NMR spectral pattern indicates ¹H NMR (CDCl₃, 400 MHz): δ 6.99-6.90 (m, 2H), 4.83 (s, 2H), 4.48 (dd, J=4, 2.8 Hz, 1H), 3.80 (s, 3H), 3.39 (q, J=7.2 Hz, 2H), 3.26 (q, J=7.2 Hz, 2H), 2.88 (dd, J=3.6, 12.8 Hz, 1H), 2.76 (dd, J=7.2, 9.2 Hz, 1H), 1.24 (t, J=7.2 Hz, 3H), 1.14 (t, J=7.2 Hz, 3H) for the (N,N-Diethylcarbamoyl)methyl methyl (2E)but-2-ene-1,4-dioate:malic acid cocrystal.

FIG. 15 is a DSC thermogram of the (N,N-Diethylcarbamoyl)methyl methyl (2E)but-2-ene-1,4-dioate:malic acid cocrystal. The thermogram shows the cocrystal has a melting

point of about 63° C.

The (N,N-Diethylcarbamoyl)methyl methyl (2E)but-2-ene-1,4-dioate:malic acid cocrystal is expected to have a good toxicology profile, since malic acid is known to be safe and appears on the GRAS list from the U.S. Food and Drug Administration.

(N,N-Diethylcarbamoyl)methyl methyl (2E)but-2-ene-1,4-dioate: Citric Acid Cocrystal

Another cocrystal disclosed herein is a cocrystal of (N,N-Diethylcarbamoyl)methyl methyl (2E)but-2-ene-1,4-dioate and citric acid. HPLC data indicates that the stoichiometry of (N,N-Diethylcarbamoyl)methyl methyl (2E)but-2-ene-1,4-dioate to citric acid is 1:1. Differential scanning calorimetry (DSC) analysis of this cocrystal shows a melting point between about 71° C. and about 75° C., in certain embodiments between about 72° C. and about 74° C., and in certain embodiments at about 73° C.

FIG. 16 is an X-ray powder diffractogram showing the diffraction pattern measured using Cu-K_{α} radiation of the (N,N-Diethylcarbamoyl)methyl methyl (2E)but-2-ene-1,4-dioate:citric acid cocrystal. Table 7 lists the approximate numerical values of the XRPD peak positions of the FIG. 16 diffractogram. While the entire diffractogram of FIG. 16 can be used to characterize the cocrystal, the cocrystal can also be accurately characterized with a subset of that data. For example, the peak at about 6.2 °2θ in FIG. 16 is more than 0.4 °2θ away from any peak in the X-ray powder diffraction pattern of (N,N-Diethylcarbamoyl)methyl methyl (2E)but-2-ene-1,4-dioate. In addition, the X-ray diffraction pattern of citric acid show that no citric acid peak occurs within 0.4 °2θ of 6.2 °2θ. Thus, the XRPD peak at 6.2 °2θ characterizes the (N,N-Diethylcarbamoyl)methyl methyl (2E)but-2-ene-1,4-dioate:citric acid cocrystal.

TABLE 7
XRPD Peaks for (N,N-Diethylcarbamoyl)methyl methyl
(2E)but-2-ene-1,4-dioate:citric acid cocrystal

| | | | | |
|---|---|---|---|---|
| Pos. | | | | |
| [°2Th.] | Height [cts] | FWHM [°2Th.] | d-spacing [Å] | Rel. Int. [%] |

Peaks (More Characteristic)

| | | | | |
|---|---|---|---|---|
| 6.9 | 2506.5 | 0.1 | 12.7 | 100.0 |
| 25.0 | 1148.0 | 0.1 | 3.6 | 17.6 |
| 6.2 | 959.0 | 0.1 | 14.4 | 14.7 |
| 19.1 | 921.6 | 0.1 | 4.7 | 14.2 |
| 13.9 | 877.8 | 0.1 | 6.4 | 13.5 |

Peaks (All)

| | | | | |
|---|---|---|---|---|
| 6.2 | 959.0 | 0.1 | 14.4 | 14.7 |
| 6.9 | 6506.5 | 0.1 | 12.7 | 100.0 |
| 9.8 | 245.4 | 0.1 | 9.0 | 3.8 |
| 12.3 | 604.9 | 0.1 | 7.2 | 9.3 |
| 13.4 | 799.7 | 0.1 | 6.6 | 12.3 |
| 13.9 | 877.8 | 0.1 | 6.4 | 13.5 |
| 14.5 | 79.7 | 0.1 | 6.1 | 1.2 |
| 18.6 | 704.8 | 0.1 | 4.8 | 10.8 |
| 19.1 | 921.6 | 0.1 | 4.7 | 14.2 |
| 19.3 | 120.4 | 0.1 | 4.6 | 1.9 |
| 19.7 | 73.5 | 0.1 | 4.5 | 1.1 |
| 20.6 | 261.1 | 0.1 | 4.3 | 4.0 |
| 21.2 | 142.1 | 0.1 | 4.2 | 2.2 |
| 21.9 | 161.1 | 0.1 | 4.1 | 2.5 |
| 22.6 | 127.4 | 0.1 | 3.9 | 2.0 |
| 23.1 | 50.6 | 0.1 | 3.9 | 0.8 |
| 24.8 | 462.8 | 0.1 | 3.6 | 7.1 |
| 25.0 | 1148.0 | 0.1 | 3.6 | 17.6 |
| 25.6 | 45.2 | 0.2 | 3.5 | 0.7 |
| 26.4 | 582.5 | 0.1 | 3.4 | 9.0 |
| 27.7 | 45.2 | 0.1 | 3.2 | 0.7 |
| 28.1 | 45.7 | 0.1 | 3.2 | 0.7 |
| 28.8 | 56.2 | 0.1 | 3.1 | 0.9 |
| 29.4 | 48.1 | 0.1 | 3.0 | 0.7 |
| 30.0 | 214.8 | 0.1 | 3.0 | 3.3 |
| 30.7 | 62.8 | 0.1 | 2.9 | 1.0 |
| 31.2 | 646.3 | 0.1 | 2.9 | 9.9 |
| 33.0 | 86.9 | 0.1 | 2.7 | 1.3 |
| 35.1 | 100.6 | 0.1 | 2.6 | 1.6 |
| 35.7 | 73.3 | 0.1 | 2.5 | 1.1 |

In another example, the XRPD peak at about 13.4 °2θ in FIG. 16 is more than 0.4 °2θ away from any peak in the X-ray powder diffraction pattern of (N,N-Diethylcarbamoyl)methyl methyl (2E)but-2-ene-1,4-dioate. In addition, the X-ray diffraction pattern of citric acid show that no citric acid peak occurs within 0.4 °2θ of 13.4 °2θ. Thus, the XRPD peak at 13.4 °2θ is another peak that alone or together with the peak at 6.2 °2θ characterizes the (N,N-Diethylcarbamoyl)methyl methyl (2E)but-2-ene-1,4-dioate:citric acid cocrystal.

Likewise, no XRPD peak of (N,N-Diethylcarbamoyl)methyl methyl (2E)but-2-ene-1,4-dioate or the citric acid diffraction pattern appears within 0.4 η2θ of 18.6 °2θ in FIG. 16, which is a peak in the (N,N-Diethylcarbamoyl)methyl methyl (2E)but-2-ene-1,4-dioate:citric acid cocrystal. Thus, the XRPD peak at 18.6 °2θ is another peak that alone or together with the peaks at 6.2 °2θ and/or 13.4 °2θ characterizes the (N,N-Diethylcarbamoyl)methyl methyl (2E)but-2-ene-1,4-dioate:citric acid cocrystal.

Likewise, no XRPD peak of (N,N-Diethylcarbamoyl)methyl methyl (2E)but-2-ene-1,4-dioate or the citric acid diffraction pattern appears within 0.4 °2θ of 9.8 °2θ in FIG. 16, which is a peak in the (N,N-Diethylcarbamoyl)methyl methyl (2E)but-2-ene-1,4-dioate:citric acid cocrystal. Thus, the XRPD peak at 9.8 °2θ is another peak that alone or together with the peaks at 6.2 °2θ, 3.4 °2θ and/or 18.6 °2θ characterizes the (N,N-Diethylcarbamoyl)methyl methyl (2E)but-2-ene-1,4-dioate:citric acid cocrystal.

In certain embodiments, the (N,N-Diethylcarbamoyl)methyl methyl (2E)but-2-ene-1,4-dioate:citric acid cocrystal exhibits characteristic scattering angles (2θ) at least at 6.2±0.2°, 6.9±0.2°, 13.9±0 2°l, 19.1±0.2° and 25.0±0.2° in an X-ray powder diffraction pattern measured using Cu-K_{α} radiation.

In certain embodiments, the (N,N-Diethylcarbamoyl)methyl methyl (2E)but-2-ene-1,4-dioate:citric acid cocrystal exhibits characteristic scattering angles (2θ) at least at 6.2±0.2°, 6.9±0.2°, 12.3±0.2°, 13.4±0.2°, 13.9±0.2°, 18.6±0.2°, 19.1±0.2°, 25.0±0.2°, 28.4±0.2° and 31.2±0.2° in an X-ray powder diffraction pattern measured using Cu-K_{α} radiation.

In certain embodiments, the (N,N-Diethylcarbamoyl)methyl methyl (2E)but-2-ene-1,4-dioate:citric acid cocrystal exhibits characteristic scattering angles (2θ) at least at 6.2±0.2°, 6.9±0.2°, 9.8±0.2°, 12.3±0.2°, 13.4±0.2°, 13.9±0.2°, 18.6±0.2°, 19.1±0.2°, 20.6±0.2°, 21.9±0.2°, 24.8±0.2620 , 25.0±0.2°, 26.4±0.2°, 30.0±0.2° and 31.2±0.2° in an X-ray powder diffraction pattern measured using Cu-_{α} radiation. in certain embodiments, the (N,N-Diethylcarbamoyl)methyl methyl (2E)but-2-ene-1,4-dioate:citric acid cocrystal exhibits characteristic scattering angles (2θ) at least at 6.2±0.1°, 6.9±0.1°, 13.9±0.1°, 19.1±0.1° and 25.0±0.1° in an X-ray powder diffraction pattern measured using Cu-K_{α} radiation.

In certain embodiments, the (N,N-Diethylcarbamoyl)methyl methyl (2E)but-2-ene-1,4-dioate:citric acid cocrystal exhibits characteristic scattering angles (2θ) at least at 6.2±0.1°, 6.9±0.1°, 12.3±0.1°, 13.4±0.1°, 13.9±0.1°, 18.6±0.1°, 19.1±0.1°, 25.0±0.1°, 26.4±0.1° and 31.2±0.1° in an X-ray powder diffraction pattern measured using Cu-_{α} radiation.

In certain embodiments, the (N,N-Diethylcarbamoyl)methyl methyl (2E)but-2-ene-1,4-dioate:citric acid cocrystal exhibits characteristic scattering angles (2θ) at least at 6.2±0.1°, 6.9±0.1°, 9.8±0.1°, 12.3±0.1°, 13.4±0.1°, 13.9±0.1°, 18.6±0.1°, 19.1±0.1°, 20.6±0.1°, 21.9±0.1°, 24.8±0.1°, 25.0±0.1°, 26.4±0.1°, 30.0±0.1° and 31.2±0.1° in an X-ray powder diffraction pattern measured using Cu-K_{α} radiation.

FIG. 17 is a spectrogram showing the NMR spectrum of the (N,N-Diethylcarbamoyl)methyl methyl (2E)but-2-ene-1,4-dioate:citric acid cocrystal. The NMR spectral pattern indicates ¹H NMR (CDCl₃, 400 MHz): δ 6.99-6.90 (m, 2H), 4.83 (s, 2H), 3.80 (s, 3H), 3.39 (q, J=7.2 Hz, 2H), 3.26 (q, J=7.2 Hz, 2H), 2.86 (d, J=15 Hz, 2H), 2.76 (d, J=15 Hz, 2H), 1.24 (t, J=7.2 Hz, 3H), 1.14 ft, J=7.2 Hz, 3H) for the (N,N-Diethylcarbamoyl)methyl methyl (2E)but-2-ene-1,4-dioate:citric acid cocrystal.

FIG. 18 is a DSC thermogram of the (N,N-Diethylcarbamoyl)methyl methyl (2E)but-2-ene-1,4-dioate:citric acid cocrystal. The thermogram shows the cocrystal has a melting point of about 73° C.

The (N,N-Diethylcarbamoyl)methyl methyl (2E)but-2-ene-1,4-dioate:citric acid cocrystal is expected to have a good toxicology profile, since citric acid is known to be safe and appears on the GRAS list from the U.S. Food and Drug Administration.

Pharmaceutical Compositions

In various aspects, the present disclosure relates to pharmaceutical compositions comprising a therapeutically effective amount of a cocrystal disclosed herein and a pharmaceutically acceptable carrier (also known as a pharmaceutically acceptable excipient). The cocrystals disclosed herein have the same pharmaceutical activity as their respective active pharmaceutical ingredient (API), namely, methyl hydrogen fumarate (MHF). Pharmaceutical compositions for the treatment of any one or more diseases and disorders contain a therapeutically effective amount of a cocrystal disclosed herein as appropriate for treatment of a patient with the particular disease(s) or disorders).

A “therapeutically effective amount” of a disclosed cocrystal (discussed here concerning the pharmaceutical compositions) refers to an amount sufficient to reduce the effects of an inflammatory or autoimmune response or disorder. The actual amount required for treatment of any particular patient will depend upon a variety of factors including the disorder being treated and its severity; the specific pharmaceutical composition employed; the age, body weight, general health, sex and diet of the patient; the mode of administration; the time of administration; the route of administration; the rate of excretion of a disclosed cocrystal; the duration of the treatment; any drugs used in combination or coincidental with the specific compound employed; the discretion of the prescribing physician; and other such factors well known in the art. These factors are discussed in Goodman and Oilman's “The Pharmacological Basis of Therapeutics”, Tenth Edition, A. Oilman, J. Hardman and L. Limbird, eds., McGraw-Hill Press, 155-173, 2001.

A pharmaceutical composition may be any pharmaceutical form which maintains the crystalline form of a disclosed cocrystal. In certain embodiments, the pharmaceutical composition may be selected from a solid form, a liquid suspension, an injectable composition, a topical form, and a transdermal form.

Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. For a pharmaceutical composition comprising a cocrystal disclosed herein, a carrier should be chosen that maintains the cocrystal. In other words, the carrier should not substantially alter the crystalline form of the cocrystal. For example, a liquid carrier which would dissolve the cocrystal should not be used. Nor should the carrier be otherwise incompatible with a cocrystal, such as by producing any undesirable biological effect or otherwise interacting in a deleterious manner with any other component(s) of the pharmaceutical composition.

In some embodiments, the pharmaceutical compositions are formulated in unit dosage forms for ease of administration and uniformity of dosage. A “unit dosage form” refers to a physically discrete unit of therapeutic agent appropriate for the patient to be treated. It will be understood, however, that the total daily dosage of a cocrystal and its pharmaceutical compositions will typically be decided by the attending physician within the scope of sound medical judgment.

Because the crystalline form of a cocrystal disclosed herein is more easily maintained during their preparation, solid dosage forms may be employed in numerous embodiments for the pharmaceutical compositions. In some embodiments, solid dosage forms for oral administration include capsules, tablets, pills, powders, and granules. In such solid dosage forms, the active compound is mixed with at least one inert, pharmaceutically acceptable carrier such as sodium citrate or dicalcium phosphate. The solid dosage form may also include one or more of: a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol: d) disintegrating agents such as agar-agar, calcium carbonate, potato or tapioca starch, alginic acid, certain silicates, and sodium carbonate; e) dissolution retarding agents such as paraffin; f) absorption accelerators such as quaternary ammonium compounds; g) wetting agents such as, tor example, cetyl alcohol and glycerol monostearate; h) absorbents such as kaolin and bentonite clay; and i) lubricants such as talc, calcium stearate, magnesium stearate, solid polyethylene glycols, sodium lauryl sulfate. The solid dosage forms may also comprise buffering agents. They may optionally contain opacifying agents and can also be of a composition such that they release the active ingredient(s) only in a certain part of the intestinal tract, optionally, in a delayed manner. Remington's Pharmaceutical Sciences, Sixteenth Edition. E. W. Martin (Mack Publishing Co., Easton, Pa., 1980) discloses various carriers used in formulating pharmaceutical compositions and known techniques for the preparation thereof. Solid dosage forms of pharmaceutical compositions can also be prepared with coatings and shells such as enteric coatings and other coatings well known in the pharmaceutical formulating art.

A cocrystal disclosed herein can be in a solid micro-encapsulated form with one or more carriers as discussed above, Microencapsulated forms of a cocrystal may also be used in soft and hard-filled gelatin capsules with carriers such as lactose or milk sugar as well as high molecular weight polyethylene glycols and the like.

Also disclosed herein are methods for the treatment of the disorders disclosed herein. The cocrystals, and pharmaceutical compositions comprising them, may be administered using any amount, any form of pharmaceutical composition and any route of administration effective for the treatment. After formulation with an appropriate pharmaceutically acceptable carrier in a desired dosage, as known by those of skill in the art, the pharmaceutical compositions can be administered to humans and other animals orally, rectally, parenterally, intravenously, intracisternally, intravaginally, intraperitoneally, topically (as by powders, ointments, or drops), bucally, as an oral or nasal spray, or the like, depending on the location and severity of the condition being treated. In certain embodiments, the cocrystals may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject.

Therapeutic Uses

The (N,N-Diethylcarbamoyl)methyl methyl (2E)but-2-ene-1,4-dioate cocrystals disclosed herein may be used to treat diseases, disorders, conditions, and/or symptoms of any disease or disorder for which MHF is known to provide, or is later found to provide, therapeutic benefit. MHF is known to be effective in treating psoriasis, multiple sclerosis, an inflammatory bowel disease, asthma, chronic obstructive pulmonary disease, and arthritis. Hence, the (N,N-Diethylcarbamoyl)methyl methyl (2E)but-2-ene-1,4-dioate cocrystals disclosed herein may also be used to treat any one or more of the foregoing diseases and disorders. In some embodiments, the (N,N-Diethylcarbamoyl)methyl methyl (2E)but-2-ene-1,4-dioate cocrystals disclosed herein may be used to treat psoriasis. In some embodiments, the (N,N-Diethylcarbamoyl)methyl methyl (2E)but-2-ene-1,4-dioatecocrystals disclosed herein may be used to treat multiple sclerosis. In some embodiments, the (N,N-Diethylcarbamoyl)methyl methyl (2E)but-2-ene-1,4-dioatecocrystals disclosed herein may be used to treat alopecia areata. The underlying etiology of any of the foregoing diseases being treated may have a multiplicity of origins.

Further, in certain embodiments, a therapeutically effective amount of one or more of the (N,N-Diethylcarbamoyl)methyl methyl (2E)but-2-ene-1,4-dioate cocrystals may be administered to a patient, such as a human, as a preventative measure against various diseases or disorders. Thus, a therapeutically effective amount of one or more of the (N,N-Diethylcarbamoyl)methyl methyl (2E)but-2-ene-1,4-dioate cocrystals may be administered as a preventative measure to a patient having a predisposition for and/or history of immunological, autoimmune, and/or inflammatory diseases including psoriasis, asthma and chronic obstructive pulmonary diseases, cardiac insufficiency including left ventricular insufficiency, myocardial infarction and angina pectoris, mitochondrial and neurodegenerative diseases (such as Parkinson's disease, Alzheimer's disease, Huntington's disease, retinopathia pigmentosa and mitochondrial encephalomyopathy), transplantation rejection, autoimmune diseases including multiple sclerosis, ischemia and reperfusion injury, AGE-induced genome damage, inflammatory bowel diseases such as Crohn's disease and ulcerative colitis; and NF-kB mediated diseases.

Psoriasis

Psoriasis is characterized by hyperkeratosis and thickening of the epidermis as well as by increased vascularity and infiltration of inflammatory cells in the dermis. Psoriasis vulgaris manifests as silvery, scaly, erythematous plaques on typically the scalp, elbows, knees, and buttocks. Guttate psoriasis occurs as tear-drop sized lesions.

Fumaric acid esters are recognized for the treatment of psoriasis and dimethyl fumarate is approved for the systemic treatment of psoriasis in Germany (Mrowietz and Asadullah, Trends Mol Med (2005), 11(1): 43-48; and Mrowietz et al., Br J Dermatology (1999), 141: 424-429).

Efficacy of the (N,N-Diethylcarbamoyl)methyl methyl (2E)but-2-ene-1,4-dioate cocrystals for treating psoriasis can be determined using animal models and in clinical trials.

Inflammatory Arthritis

Inflammatory arthritis includes diseases such as rheumatoid arthritis, juvenile rheumatoid arthritis (juvenile idiopathic arthritis), psoriatic arthritis, and ankylosing spondylitis, among others. The pathogenesis of immune-mediated inflammatory diseases including inflammatory arthritis is believed to involve TNF and NK-kB signaling pathways (Tracey et al., Pharmacology & Therapeutics (2003), 117: 244-279). Dimethyl fumarate has been shown to inhibit TNF and inflammatory diseases, including inflammatory arthritis, are believed to involve TNF and NK-kB signaling. Therefore, dimethyl fumarate may be useful in treating inflammatory arthritis (Lowewe et al., J Immunology (2002), 168; 4781-4787).

The efficacy of the (N,N-Diethylcarbamoyl)methyl methyl (2E)but-2-ene-1,4-dioate cocrystals for heating inflammatory arthritis can be determined using animal models and in clinical trials.

Multiple Sclerosis

Multiple sclerosis (MS) is an inflammatory autoimmune disease of the central nervous system caused by an autoimmune attack against the insulating axonal myelin sheaths of the central nervous system. Demyelination leads to the breakdown of conduction and to severe disease with destruction of local axons and irreversible neuronal cell death. The symptoms of MS are highly varied, with each individual patient exhibiting a particular pattern of motor, sensible, and sensory disturbances. MS is typified pathologically by multiple inflammatory foci, plaques of demyelination, gliosis, and axonal pathology within the brain and spinal cord, all of which contribute to the clinical manifestations of neurological disability (see e.g., Wingerchuk, Lab Invest (2001), 81: 263-281; and Virley, NeuroRx (2005), 2(4): 638-649). Although the causal events that precipitate MS are not fully understood, evidence implicates an autoimmune etiology together with environmental factors, as well as specific genetic predispositions. Functional impairment, disability, and handicap are expressed as paralysis, sensory and octintive disturbances, spasticity, tremor, a lack of coordination, and visual impairment, any one of which negatively impacts the quality of life of the individual. The clinical course of MS can vary from individual to individual, but invariably the disease can be categorized in three forms relapsing-remitting, secondary progressive, and primary progressive.

Studies support the efficacy of fumaric acid esters for treating MS and fumaric acid esters are presently undergoing phase III clinical testing for such treatment (Schimrigk et al., Eur J Neurology (2006), 13: 604-610; and Wakkee and Thio, Current Opinion Investigational Drugs (2007), 8(11): 955-962).

Assessment of MS treatment efficacy in clinical trials can be accomplished using tools such as the Expanded Disability Status Scale and the MS Functional, as well as magnetic resonance imaging, lesion load, biomarkers, and self-reported quality of life. Animal models of MS shown to be useful to identify and validate potential therapeutics include experimental autoimmune/allergic encephalomyelitis (EAE) rodent models that simulate the clinical and pathological manifestations of MS and nonhuman primate EAE models.

The efficacy of the (N,N-Diethylcarbamoyl)methyl methyl (2E)but-2-ene-1,4-dioate cocrystals for treating MS can be determined using animal models and in clinical trials.

Inflammatory Bowel Disease (Crohn's Disease, Ulcerative Colitis)

Inflammatory bowel disease (IBD) is a group of Inflammatory conditions of the large intestine, and in some cases the small intestine, that includes Crohn's disease and ulcerative colitis. Crohn's disease, which is characterized by areas of inflammation with areas of normal lining in between, can affect any part of the gastrointestinal tract from the mouth to the anus. The main gastrointestinal symptoms are abdominal pain, diarrhea, constipation, vomiting, weight loss, and/or weight gain. Crohn's disease can also cause skin rashes, arthritis, and inflammation of the eye. Ulcerative colitis is characterized by ulcers or open sores in the large intestine or colon. The main symptom of ulcerative colitis is typically constant diarrhea with mixed blood of gradual onset. Othei types of intestinal bowel disease include collagenous colitis, lymphocytic colitis, ischaemic colitis, division colitis, Behcet's colitis and indeterminate colitis.

Fumaric acid esters are inhibitors of NF-kB activation and therefore may be useful in treating inflammatory diseases such as Crohn's disease and ulcerative colitis (Atreya et al., J Intern Med (2008), 263(8): 591-596).

The efficacy of the (N,N-Diethylcarbamoyl)methyl methyl (2E)but-2-ene-1,4-dioate cocrystals for treating inflammatory bowel disease can be evaluated using animal models and in clinical trials. Useful animal models of inflammatory bowel disease are known.

Asthma

Asthma is reversible airway obstruction in which the airway occasionally constricts, becomes inflamed, and is lined with an excessive amount of mucus. Symptoms of asthma include dyspnea, wheezing, chest tightness, and cough. Asthma episodes may be induced by airborne allergens, food allergies, medications, inhaled irritants, physical exercise, respiratory infection, psychological stress, hormonal changes, cold weather, or other factors.

As an inhibitor of NF-kB activation and as shown In animal studies (Joshi et al., U.S. Patent Application Publication No. 2007/0027076) fumaric acid esters may be useful in treating pulmonary diseases such as asthma and chronic obstructive pulmonary disorder.

The efficacy of the (N,N-Diethylcarbamoyl)methyl methyl (2E)but-2-ene-1,4-dioate cocrystals for treating asthma can be assessed using animal models and in clinical trials.

Chronic Obstructive Pulmonary Disease

Chronic obstructive pulmonary disease (COPD), also known as chronic obstructive airway disease, is a group of diseases characterized by the pathological limitation of airflow in the airway that is not fully reversible, and includes conditions such as chronic bronchities, emphysema, as well as other lung disorders such as asbestosis, pneumoconiosis, and pulmonary neoplasms (see, e.g., Barnes, Pharmacological Reviews (2004), 56(4): 515-548). The airflow limitation is usually progressive and associated with an abnormal inflammatory response of the lungs to noxious particles and gases. COPD is characterized by a shortness of breath that can last for months or years, possibly accompanied by wheezing, and a persistent cough with sputum production. COPD is most often caused by tobacco smoking, although it can also be caused by other airborne irritants such as coal dust, asbestos, urban pollution, or solvents. COPD encompasses chronic obstructive bronchiolitis with fibrosis and obstruction of small airways, and emphysema with enlargement of airspaces and destruction of lung parenchyma, loss of lung elasticity, and closure of small airways.

The efficacy of administering the (N,N-Diethylcarbamoyl)methyl methyl (2E)but-2-ene-1,4-dioate cocrystals for treating chronic obstructive pulmonary disease may be assessed using animal models of chronic obstructive pulmonary disease and in clinical studies. For example, murine models of chronic obstructive pulmonary disease are known.

Neurodegenerative Disorders

Neurodegenerative diseases such as Parkinson's disease, Alzheimer's disease, Huntington's disease and amyoptrophic lateral sclerosis are characterized by progressive dysfunction and neuronal death. NF-kB inhibition has been proposed as a therapeutic target for neurodegenerative diseases (Camandola and Mattson, Expert Opin Ther Targets (2007), 11(2): 123-32).

Parkinson's Disease

Parkinson's disease is a slowly progressive degenerative disorder of the nervous system characterized by tremor when muscles are at rest (resting tremor), slowness of voluntary movements, and increased muscle tone (rigidity). In Parkinson's disease, nerve cells in the basal ganglia (e.g., the substantia nigra) degenerate, and thereby reduce the production of dopamine and the number of connections between nerve cells in the basal ganglia. As a result, the basal ganglia are unable to control smooth muscle movements and coordinate changes in posture as normal, leading to tremor, incoordination, and slowed, reduced movement (bradykinesia) (Blandini, et al., Mol. Neurobiol. (1996), 12: 73-94).

The efficacy of the (N,N-Diethylcarbamoyl)methyl methyl (2E)but-2-ene-1,4-dioate cocrystals for treating Parkinson's disease may be assessed using animal and human models of Parkinson's disease and in clinical studies.

Alzheimer's Disease

Alzheimer's disease is a progressive loss of mental function characterized by degeneration of brain tissue, including loss of nerve cells and the development of senile plaques and neurofibrillary tangles. In Alzheimer's disease, parts of the brain degenerate, destroying nerve cells and reducing the responsiveness of the maintaining neurons to neurotransmitters. Abnormalities in brain tissue consist of senile or neuritis plaques (e.g., clumps of dead nerve ceils containing an abnormal, insoluble protein called amyloid) and neurofibrillary tangles, twisted strands of insoluble proteins in the nerve cell.

The efficacy of the (N,N-Diethylcarbamoyl)methyl methyl (2E)but-2-ene-1,4-dioate cocrystals for treating Alzheimer's disease may be assessed using animal and human models of Alzheimer's disease and in clinical studies.

Huntington's Disease

Huntington's disease is an autosomal dominant neurodegenerative disorder in which specific cell death occurs in the neostriatum and cortex (Martin, N Engl J Med (1999), 340: 1970-80). Onset usually occurs during the fourth or fifth decade of life, with a mean survival at age of onset of 14 to 20 years. Huntington's disease is universally fatal, and there is no effective treatment. Symptoms include a characteristic movement disorder (Huntington's chorea), cognitive dysfunction, and psychiatric symptoms. The disease is caused by a mutation encoding an abnormal expansion of CAG-encoded polyglutamine repeats in the protein, huntingtin.

The efficacy of the (N,N-Diethylcarbamoyl)methyl methyl (2E)but-2-ene-1,4-dioate cocrystals for treating Huntington's disease may be assessed using animal and human models of Huntington's disease and in clinical studies.

Amyotrophic Lateral Sclerosis

Amyotrophic lateral sclerosis (ALS) is a progressive neurodegenerative disorder characterized by the progressive and specific loss of motor neurons in the brain, brain stem, and spinal cord (Rowland and Schneider, N Engl J Med (2001), 344: 1688-1700), ALS begins with weakness, often in the hands and less frequently in the feet that generally progresses up an arm or leg. Overtime, weakness increases and spasticity develops characterized by muscle twitching and tightening, followed by muscle spasms and possibly tremors. The average age of onset is 55 years, and the average life expectancy after the clinical onset is 4 years. The only recognized treatment for ALS is riluzole, which can extend survival by only about three months.

The efficacy the (N,N-Diethylcarbamoyl)methyl methyl (2E)but-2-ene-1,4-dioate cocrystals for treating ALS may be assessed using animal and human models of ALS and in clinical studies.

Alopecia Areata

The efficacy the (N,N-Diethylcarbamoyl)methyl methyl (2E)but-2-ene-1,4-dioate cocrystals for treating alopecia areata may be assessed using animal and human models of alopecia areata and in clinical studies.

Other Diseases

Other diseases and conditions for which the (N,N-Diethylcarbamoyl)methyl methyl (2E)but-2-ene-1,4-dioate cocrystals can be useful in treating include: rheumatica, granuloma annulare, lupus, autoimmune carditis, eczema, sarcoidosis, autoimmune diseases including acute disseminated encephalomyelitis, Addison's disease, alopecia areata, ankylosing spondylitis, antiphospholipid antibody syndrome, autoimmune hemolytic anemia, autoimmune hepatitis, autoimmune inner ear disease, bullous pemphigoid, Behcet's disease, celiac disease, Chagas disease, chronic obstructive pulmonary disease, Crohn's disease, dermatomyositis, diabetes mellitus type I, endometriosis, Goodpasture's syndrome, Graves' disease, Guillain-Barre syndrome Hashimoto's disease, hidradenitis suppurativea, Kawasaki disease, IgA neuropathy, idiopathic thrombocytopenic purpura, interstitial cystitis, lupus erythematosus, mixed connective tissue disease, morphea, multiple sclerosis, myasthenia gravis, narcolepsy, neuromyotonia, pemphigus vulgaris, pernicious anaemia, psoriasis, psoriatic arthritis, polymyositis, primary biliary cirrhosis, rheumatoid arthritis, schizophrena, scleroderma, Sjogren's syndrome, stiff person syndrome, temporal arteritis, ulcerative colitis, vasculitis, vitiligo, Wegener's granulomatosis, optic neuritis, neuromyelitis optica, subacute necrotizing myelopathy, balo concentric sclerosis, transverse myelitis, susac syndrome, central nervous system vasculitis, neurosarcoidosis, Chareott-Marie-Tooth Disease, progressive supranuclear palsy, neurodegeneration with brain iron accumulation, paraneoplastic syndromes, primary lateral sclerosis, Alper's Disease, monomelic myotrophy, adrenal leukodystrophy, Alexanders Disease, Canavan disease, childhood ataxia with central nervous system hypomyelination, Krabbe Disease, Pelizaeus-Merzbacher disease, Schilders Disease, Zellweger's syndrome, Sjorgren's Syndrome, human immunodeficiency viral infection, hepatitis C viral infection, herpes simplex viral infection and a tumor.

### EXAMPLES

The following examples are included to demonstrate certain embodiments of the present disclosure. It should be appreciated by those of skill in the art that the techniques disclosed in the examples which follow represent techniques discovered by the inventors to function well in the practice of the subject matter of the present disclosure, and thus can be considered to constitute modes for its practice. However, those of skill in the art should, in light of the present disclosure, appreciate that many changes can be made to the specific embodiments disclosed herein and still obtain a like or similar result without departing from the spirit and scope of the subject matter of the present disclosure.

### Example 1

### Synthesis, Purification and Analysis of Cocrystal of (N,N-Diethylcarbamoyl)methyl methyl (2E)but-2-ene-1,4-dioate and Urea

Cocrystals of (N,N-Diethylcarbamoyl)methyl methyl (2E)but-2-ene-1,4-dioate and urea were prepared as follows. First, 4.00 g of urea was dissolved in 10 ml of water. Then 2.44 g of (N,N-Diethylcarbamoyl)methyl methyl (2E)but-2-ene-1,4-dioate was added to the solution. The resulting suspension was briefly sonicated, and was allowed to stir for 24 hours at room temperature. The starting concentrations of (N,N-Diethylcarbamoyl)methyl methyl (2E)but-2-ene-1,4-dioate and urea were chosen such that the thermodynamically stable solid phase at equilibrium is the cocrystal of (N,N-Diethylcarbamoyl)methyl methyl (2E)but-2-ene-1,4-dioateand urea. The product was collected and dried through vacuum filtration to yield the (N,N-Diethylcarbamoyl)methyl methyl (2E)but-2-ene-1,4-dioate:urea (1:1) cocrystal. The measured melting points were 58° C. for (N,N-Diethylcarbamoyl)methyl methyl (2E)but-2-ene-1,4-dioate, 135° C. for urea, and 77° C. G for the cocrystal.

Differential Scanning Calorimetry (DSC) Analysis

The DSC analysis was conducted using the TA Instruments Q2000 DSC equipped with a refrigerated cooling system. For all DSC analysis, 2-5 mg of sample was loaded into T_{Zero} aluminum pans with crimpled lids. A pinhole was made at the center of the lid to avoid any pressure buildup during heating. Samples were equilibrated at 10° C. and ramped to 180° C. at a rate of 10° C. per minute under a purge of dry nitrogen gas, The data acquisition was controlled by Thermal Advantage software Release 4.9.1. The data were analyzed with Universal Analysis 2000 software (version 4.5 A).

The DSC thermogram (FIG. 3) shows that the (N,N-Diethylcarbamoyl)methyl methyl (2E)but-2-ene-1,4-dioate:urea cocrystal first melts at about 77° C., which is significantly higher than the melting point of crystalline (N,N-Diethylcarbamoyl)methyl methyl (2E)but-2-ene-1,4-dioate. The melting points of (N,N-Diethylcarbamoyl)methyl methyl (2E)but-2-ene-1,4-dioate and urea are 58° C. and 135° C., respectively. Hot-stage microscopy study shows that melting of the cocrystal and the crystallization of urea occurs simultaneously. The second melting transition with onset temperature at 129° C. corresponds roughly to the melting point of urea, which has a melting point of about 133° C.-135° C.

Thermogravimetric Analysis (TGA)

The thermal gravimetric analysis was conducted using a TA Instruments Q5000 thermogravimetric analyzer. For all TGA analysis, 5-10 mg of sample was loaded to a platinum pan and was heated to 180° C. at a rate of 10° C. per minute under a purge of dry nitrogen gas. The data acquisition was controlled by Thermal Advantage software Release 4.9.1. The data was analyzed with Universal Analysis 2000 software (version 4.5 A).

The TGA thermogram shows that the (N,N-Diethylcarbamoyl)methyl methyl (2E)but-2-ene-1,4-dioate:urea cocrystal does not undergo any weight loss prior to melting, which indicates that the cocrystal is an anhydrous crystalline solid.

X-ray Powder Diffraction (XRPD) Analysis

Powder X-ray diffraction analysis was performed using the PANalytical X'Pert Pro X-ray diffractometer. The X-ray source was Cu K_{α} radiation (λ=1.54051 Å) with output voltage of 45 kV and current of 40 mA. The instrument adopts a para-focusing Bragg-Brentano geometry with incident divergence and scattering slits set at 1/16° and ⅛° respectively. Large Seller slits (0.04 rad) were used for both incident and diffracted beam to remove axial divergence. A small amount of powder (9-12 mg) was gently pressed onto the single crystal silicon sample holder to form a smooth surface, and samples were subjected to spinning at a rate of two revolutions per second, throughout the acquisition process. The samples were scanned from 2° to 40° in 2θ with a step size of 0.017° and a scan speed of 0.067°/sec. The data acquisition was controlled and analyzed by X'Pert Data Collector (version 2.2d) and X'Pert Data Viewer (version 1.2c), respectively.

The X-ray diffraction pattern for the (N,N-Diethylcarbamoyl)methyl methyl (2E)but-2-ene-1,4-dioate:urea cocrystal is shown in FIG. 1. Unless otherwise specified, the experimental data for X-ray powder diffraction were collected at room temperature.

NMR Analysis

Proton NMR (400 MHz) NMR spectra were recorded on a Varian AS 400 NMR spectrometer equipped with an auto-sampler and data processing software. CDCl₃ (99.8% D), or MeOH-d₃ (99.8% D), were used as solvents unless otherwise noted. The CHCl₃, or MeOH-d₃ solvent signals were used for calibration of the individual spectra. The NMR spectral pattern for the (N,N-Diethylcarbamoyl)methyl methyl (2E)but-2-ene-1,4-dioate:urea cocrystal is shown in FIG. 2.

### Example 2

### Synthesis, Purification and Analysis of Cocrystal of (N,N-Diethylcarbamoyl)methyl methyl (2E)but-2-ene-1,4-dioate and fumaric Acid

Cocrystals of (N,N-Diethylcarbamoyl)methyl methyl (2E)but-2-ene-1,4-dioate and fumaric acid were prepared as follows. First, 0.44 mg of fumaric acid was dissolved in 10 ml of ethyl acetate/heptane mixture (⅓v/v). Then 2.44 g of (N,N-Diethylcarbamoyl)methyl methyl (2E)but-2-ene-1,4-dioate was added to the solution. The resulting suspension was briefly sonicated, and was allowed to stir for 24 hours at room temperature. The starting concentrations of (N,N-Diethylcarbamoyl)methyl methyl (2E)but-2-ene-1,4-dioate and fumaric acid were chosen such that the thermodynamically stable solid phase at equilibrium is the cocrystal of (N,N-Diethylcarbamoyl)methyl methyl (2E)but-2-ene-1,4-dioate and fumaric acid. The product was collected and dried through vacuum filtration to yield the (N,N-Diethylcarbamoyl)methyl methyl (2E)but-2-ene-1,4-dioate:fumaric acid (2:1) cocrystal. The measured melting points were 58° C. for (N,N-Diethylcarbamoyl)methyl methyl (2E)but-2-ene-1,4-dioate, 287° C. for fumaric acid, and 74° C. for the cocrystal.

Differential Scanning Calorimetry (DSC) Analysis

The DSC analysis was conducted using the TA Instruments Q2000 DSC equipped with a refrigerated cooling system. For all DSC analysis, 2-10 mg of sample was loaded into T_{zero} aluminum pans with crimpled lids. A pinhole was made at the center of the lid to avoid any pressure buildup during heating. Samples were equilibrated at 10° C. and ramped to 320° C. at a rate of 10° C. per minute under a purge of dry nitrogen gas. The data acquisition was controlled by Thermal Advantage software Release 4.9,1. The data were analyzed with Universal Analysis 2000 software (version 4.5A).

DSC thermogram in FIG. 6 shows that of the (N,N-Diethylcarbamoyl)methyl methyl (2E)but-2-ene-1,4-dioate:fumaric acid cocrystal melts at about 74° C. which is significantly higher than the melting point of crystalline (N,N-Diethylcarbamoyl)methyl methyl (2E)but-2-ene-1,4-dioate. The melting points of (N,N-Diethylcarbamoyl)methyl methyl (2E)but-2-ene-1,4-dioate and fumaric acid are 58° C. and 287° C., respectively.

Thermogravimetric Analysis (TGA)

The thermal gravimetric analysis was conducted using a TA Instruments G5000 thermogravimetric analyzer. For all TGA analysis, 5-10 mg of sample was loaded to a platinum pan and was heated to 320° C. at a rate of 10° C. per minute under a purge of dry nitrogen gas. The data acquisition was controlled by Thermal Advantage software Release 4.9.1. The data were analyzed with Universal Analysis 2000 software (version 4.5A).

The TGA thermogram shows that the (N,N-Diethylcarbamoyl)methyl methyl (2E)but-2-ene-1,4-dioate:fumaric acid cocrystal does not undergo any weight loss prior to melting, which indicates that the cocrystal is an anhydrous crystalline solid.

X-ray Powder Diffraction (XRPD) Analysis

Powder X-ray diffraction analysis was performed using the PANalytical X'Pert Pro X-ray diffractometer. The X-ray source was Cu K_{α} radiation (λ=1.54051 Å) with output voltage of 45 kV and current of 40 mA. The instrument adopts a para-focusing Bragg-Brentano geometry with incident divergence and scattering slits set at 1/16° and ⅛° respectively. Large Soller site (0.04 rad) were used for both incident and diffracted beam to remove axial divergence. A small amount of powder (9-12 mg) was gently pressed onto the single crystal silicon sample holder to form a smooth surface, and samples were subjected to spinning at a rate of two revolutions per second, throughout the acquisition process. The samples were scanned from 2° to 40° in 2θ with a step size of 0.017° and a scan speed of 0.067 °/sec. The data acquisition was controlled and analyzed by X'Pert Data Collector (version 2.2d) and X'Pert Data Viewer (version 1.2c), respectively.

The X-ray diffraction pattern for the (N,N-Diethylcarbamoyl)methyl methyl (2E)but-2-ene-1,4-dioate;fumaric acid cocrystal is shown in FIG. 4. Unless otherwise specified, the experimental data for X-ray powder diffraction were collected at room temperature.

NMR Analysis

Proton NMR (400 MHz) NMR spectra were recorded on a Varian AS 400 NMR spectrometer equipped with an auto-sampler and data processing software. CDCl₃ (99.8% D), or MeOH-d₃ (99.8% D), were used as solvents unless otherwise noted. The CHCl₃ or MeOH-d₃ solvent signals were used for calibration of the individual spectra. The NMR spectral pattern for the (N,N-Diethylcarbamoyl)methyl methyl (2E)but-2-ene-1,4-dioate:fumaric acid cocrystal is shown in FIG. 5.

### Example 3

### Synthesis, Purification and Analysis of Cocrystal of (N,N-Diethylcarbamoyl)methyl methyl (2E)but-2-ene-1,4-dioate and Succinic Acid

Cocrystals of (N,N-Diethylcarbamoyl)methyl methyl (2E)but-2-ene-1,4-dioate and succinic acid were prepared as follows. First, 1.18 g of succinic acid was dissolved in 10 mL of water. Then 2.44 g of (N,N-Diethylcarbamoyl)methyl methyl (2E)but-2-ene-1,4-dioate was added to the solution. The resulting suspension was briefly sonicated, and was allowed to stir for 24 hours at room temperature. The starting concentrations of (N,N-Diethylcarbamoyl)methyl methyl (2E)but-2-ene-1,4-dioate and succinic acid were chosen such that the thermodynamically stable solid phase at equilibrium is the cocrystal of (N,N-Diethylcarbamoyl)methyl methyl (2E)but-2-ene-1,4-dioate and succinic acid. The product was collected and dried through vacuum filtration to yield the (N,N-Diethylcarbamoyl)methyl methyl (2E)but-2-ene-1,4-dioate:succinic acid (2:1) cocrystal. The measured melting points were 58° C. for (N,N-Diethylcarbamoyl)methyl methyl (2E)but-2-ene-1,4-dioate, 186° C. for succinic acid, and 64° C. for the cocrystal.

Differential Scanning Calorimetry (DSC) Analysis

The DSC analysis was conducted using the TA instruments Q2000 DSC equipped with a refrigerated cooling system. For all DSC analysis, 2-10 mg of sample was loaded into T_{zero} aluminum pans with crimpled lids. A pinhole was made at the center of the lid to avoid any pressure buildup during heating. Samples were equilibrated at 10° C. and ramped to 250° C. at a rate of 10° C. per minute under a purge of dry nitrogen gas. The data acquisition was controlled by Thermal Advantage software Release 4.9.1. The data were analyzed with Universal Analysis 2000 software (version 4.5A).

DSC thermogram in FIG. 9 shows that the (N,N-Diethylcarbamoyl)methyl methyl (2E)but-2-ene-1,4-dioate:succinic add cocrystal melts at about 64° C., which is significantly higher than the melting point of crystalline (N,N-Diethylcarbamoyl)methyl methyl (2E)but-2-ene-1,4-dioate. The melting points of (N,N-Diethylcarbamoyl)methyl methyl (2E)but-2-ene-1,4-dioate and succinic acid are 58° C. and 186° C., respectively.

Thermogravimetric Analysis (TGA)

The thermal gravimetric analysis was conducted using a TA Instruments Q5000 thermogravimetric analyzer. For all TGA analysis, 5-10 mg of sample was loaded to a platinum pan and was heated to 250° C. at a rate of 10° C. per minute under a purge of dry nitrogen gas. The data acquisition was controlled by Thermal Advantage software Release 4.9.1. The data were analyzed with Universal Analysis 2000 software (version 4.5A).

The TGA thermogram shows that cocrystal of the (N,N-Diethylcarbamoyl)methyl methyl (2E)but-2-ene-1,4-dioate:succinic acid cocrystal does not undergo any weight loss prior to melting, which indicates that the cocrystal is an anhydrous crystalline solid.

X-ray Powder Diffraction (XRPD) Analysis

Powder X-ray diffraction analysis was performed using the PANalytical X'Pert Pro X-ray diffractometer. The X-ray source was Cu k_{α} radiation (λ=1.54051 Å) with output voltage of 45 kV and current of 40 mA. The instrument adopts a para-focusing Bragg-Brentano geometry with incident divergence and scattering slits set at 1/16° and ⅛° respectively. Large Soller slits (0.04 rad) were used for both incident and diffracted beam to remove axial divergence. A small amount of powder (9-12 mg) was gently pressed onto the single crystal silicon sample holder to form a smooth surface, and samples were subjected to spinning at a rate of two revolutions per second, throughout the acquisition process. The samples were scanned from 2° to 40° in 2θ with a step size of 0.017° and a scan speed of 0.067 °/sec. The data acquisition was controlled and analyzed by X'Pert Data Collector (version 2.2d) and X'Pert Data Viewer (version 1.2c), respectively.

The X-ray diffraction pattern for the (N,N-Diethylcarbamoyl)methyl methyl (2E)but-2-ene-1,4-dioate:succinic acid cocrystal is shown in FIG. 7. Unless otherwise specified, the experimental data for X-ray powder diffraction were collected at room temperature.

NMR Analysis

Proton NMR (400 MHz) NMR spectra were recorded on a Varian AS 400 NMR spectrometer equipped with an auto sampler and data processing software. CDCl₃ (99.8% D), or MeOH-d₃ (99.8% D), were used as solvents unless otherwise noted. The CHCl₃, or MeOH-d₃ solvent signals were used for calibration of the individual spectra. The NMR spectral pattern for the (N,N-Diethylcarbamoyl)methyl methyl (2E)but-2-ene-1,4-dioate;succinic acid cocrystal is shown in FIG. 8.

### Example 4

### Synthesis, Purification and Analysis of Cocrystal of (N,N-Diethylcarbamoyl)methyl methyl (2E)but-2-ene-1,4-dioate and Maleic Acid

Cocrystals of (N,N-Diethylcarbamoyl)methyl methyl (2E)but-2-ene-1,4-dioate and maleic acid were prepared as follows. First, 1.00 g of maleic acid was dissolved in 20 mL of ethyl acetate/heptane mixture (⅓ v/v). Then 2.44 g of (N,N-Diethylcarbamoyl)methyl methyl (2E)but-2-ene-1,4-dioate was added to the solution. The resulting suspension was briefly sonicated, and was allowed to stir for 24 hours at room temperature. The starting concentrations of (N,N-Diethylcarbamoyl)methyl methyl (2E)but-2-ene-1,4-dioate and maleic acid were chosen such that the thermodynamically stable solid phase at equilibrium is the cocrystal of (N,N-Diethylcarbamoyl)methyl methyl (2E)but-2-ene-1,4-dioate and maleic acid. The product was collected and dried through vacuum filtration to yield the (N,N-Diethylcarbamoyl)methyl methyl (2E)but-2-ene-1,4-dioate:maleic acid (1:1) cocrystal. The measured melting points were 58° C. for (N,N-Diethylcarbamoyl)methyl methyl (2E)but-2-ene-1,4-dioate, 131° C. for maleic acid, and 67° C. for the cocrystal.

Differential Scanning Calorimetry (DSC) Analysis

The DSC analysis was conducted using the TA Instruments Q2000 DSC equipped with a refrigerated cooling system. For all DSC analysis, 2-10 mg of sample was loaded into T_{zero} aluminum pans with crimpled lids. A pinhole was made at the center of the lid to avoid any pressure buildup during heating. Samples were equilibrated at 10° C. and ramped to 250° C. at a rate of 10° C. per minute under a purge of dry nitrogen gas. The data acquisition was controlled by Thermal Advantage software Release 4.9.1. The data were analyzed with Universal Analysis 2000 software (version 4.5A).

DSC thermogram in FIG. 12 shows that the (N,N-Diethylcarbamoyl)methyl methyl (2E)but-2-ene-1,4-dioate:maleic acid cocrystal melts at about 87° C. which is significantly higher than the melting point of crystalline (N,N-Diethylcarbamoyl)methyl methyl (2E)but-2-ene-1,4-dioate. The melting points of (N,N-Diethylcarbamoyl)methyl methyl (2E)but-2-ene-1,4-dioate and maleic acid are 58° C. and 131° C., respectively.

Thermogravimetric Analysis (TGA)

The thermal gravimetric analysis was conducted using a TA Instruments Q5000 thermogravimetric analyzer. For all TGA analysis, 5-10 mg of sample was loaded to a platinum pan and was heated to 250° C. at a rate of 10° C. per minute under a purge of dry nitrogen gas. The data acquisition was controlled by Thermal Advantage software Release 4.9.1. The data were analyzed with Universal Analysis 2000 software (version 4.5A).

The TGA thermogram shows that the (N,N-Diethylcarbamoyl)methyl methyl (2E)but-2-ene-1,4-dioate maleic acid cocrystal does not undergo any weight loss prior to melting, which indicates that the cocrystal is an anhydrous crystalline solid.

X-ray Powder Diffraction (XRPD) Analysis

Powder X-ray diffraction analysis was performed using the PANalytical X'Pert Pro X-ray diffractometer. The X-ray source was Cu k_{α} radiation (λ=1.54051 Å) with output voltage of 45 kV and current of 40 mA. The instrument adopts a para-focusing Bragg-Brentano geometry with incident divergence and scattering slits set at 1/16° and ⅛° respectively. Large Soller slits (0.04 rad) were used for both incident and diffracted beam to remove axial divergence. A small amount of powder (9-12 mg) was gently pressed onto the single crystal silicon sample holder to form a smooth surface, and samples were subjected to spinning at a rate of two revolutions per second, throughout the acquisition process. The samples were scanned from 2° to 40° in 2θ with a step size of 0.017° and a scan speed of 0.067 °/sec. The data acquisition was controlled and analyzed by X'Pert Data Collector (version 2.2d) and X'Pert Data Viewer (version 1.2c), respectively.

The X-ray diffraction pattern for the (N,N-Diethylcarbamoyl)methyl methyl (2E)but-2-ene-1,4-dioate and maleic acid cocrystal is shown in FIG. 10. Unless otherwise specified, the experimental data for X-ray powder diffraction were collected at room temperature.

NMR Analysis

Proton NMR (400 MHz) NMR spectra were recorded on a Varian AS 400 NMR spectrometer equipped with an auto-sampler and data processing software. CDCl₃ (99.8% D), or MeOH-d₃ (99.8+% D), were used as solvents unless otherwise noted. The CHCl₃, or MeOH-d₃ solvent signals were used for calibration of the individual spectra. The NMR spectral pattern for the (N,N-Diethylcarbamoyl)methyl methyl (2E)but-2-ene-1,4-dioate:maleic acid cocrystal is shown in FIG. 11.

### Example 5

### Synthesis, Purification and Analysis of Cocrystal of (N,N-Diethylcarbamoyl)methyl methyl (2E)but-2-ene-1,4-dioate and Malic Acid

Cocrystals of (N,N-Diethylcarbamoyl)methyl methyl (2E)but-2-ene-1,4-dioateand malic acid were prepared as follows. First, 0.81 g of DL-malic acid was dissolved in 40 mL of ethyl acetate/heptane mixture (⅓ v/v). Then 2.26 g of (N,N-Diethylcarbamoyl)methyl methyl (2E)but-2-ene-1,4-dioate was added to the solution. The resulting suspension was briefly sonicated, and was allowed to stir for 24 hours at room temperature. The starting concentrations of (N,N-Diethylcarbamoyl)methyl methyl (2E)but-2-ene-1,4-dioate and malic acid were chosen such that the thermodynamically stable solid phase at equilibrium is the cocrystal of (N,N-Diethylcarbamoyl)methyl methyl (2E)but-2-ene-1,4-dioate and malic acid. The product was collected and dried through vacuum filtration to yield the (N,N-Diethylcarbamoyl)methyl methyl (2E)but-2-ene-1,4-dioate:malic acid (1:1) cocrystal. The measured melting points were 58° C. for (N,N-Diethylcarbamoyl)methyl methyl (2E)but-2-ene-1,4-dioate, 131° C. for DL-malic acid, and 63° C. for the cocrystal.

Differential Scanning Calorimetry (DSC) Analysis

The DSC analysis was conducted using the TA Instruments Q2000 DSC equipped with a refrigerated cooling system. For all DSC analysis, 2-10 mg of sample was loaded into T_{zero} aluminum pans with crimpled lids. A pinhole was made at the center of the lid to avoid any pressure buildup during heating. Samples were equilibrated at 10° C. and ramped to 250° C. at a rate of 10° C. per minute under a purge of dry nitrogen gas. The data acquisition was controlled by Thermal Advantage software Release 4.9.1. The data were analyzed with Universal Analysis 2000 software (version 4.5A).

DSC thermogram in FIG. 15 shows that of the (N,N-Diethylcarbamoyl)methyl methyl (2E)but-2-ene-1,4-dioate:malic acid cocrystal melts at about 63° C., which is significantly higher than the melting point of crystalline (N,N-Diethylcarbamoyl)methyl methyl (2E)but-2-ene-1,4-dioate. The melting points of (N,N-Diethylcarbamoyl)methyl methyl (2E)but-2-ene-1,4-dioate and DL-malic acid are 58° C. and 131° C., respectively.

Thermogravimetric Analysis (TGA)

The thermal gravimetric analysis was conducted using a TA Instruments Q5000 thermogravimetric analyzer. For all TGA analysis, 5-10 mg of sample was loaded to a platinum pan and was heated to 250° C. at a rate of 10° C. per minute under a purge of dry nitrogen gas. The data acquisition was controlled by Thermal Advantage software Release 4.9.1 The data were analyzed with Universal Analysis 2000 software (version 4.5A).

The TGA thermogram shows that the (N,N-Diethylcarbamoyl)methyl methyl (2E)but-2-ene-1,4-dioate:malic acid cocrystal does not undergo any weight loss prior to melting, which indicates that the cocrystal is an anhydrous crystalline solid.

X-ray Powder Diffraction (XRPD) Analysis

Powder X-ray diffraction analysis was performed using the PANalytical X'Pert Pro X-ray diffractometer. The X-ray source was Cu k_{α} radiation (λ=1.54051 Å) with output voltage of 45 kV and current of 40 mA. The instrument adopts a para-focusing Bragg-Brentano geometry with incident divergence and scattering slits set at 1/16° and ⅛° respectively. Large Soller slits (0.04 rad) were used for both incident and diffracted beam to remove axial divergence. A small amount of powder (9-12 mg) was gently pressed onto the single crystal silicon sample holder to form a smooth surface, and samples were subjected to spinning at a rate of two revolutions per second, throughout the acquisition process. The samples were scanned from 2° to 40° in 2θ with a step size of 0.017° and a scan speed of 0.067 °/sec. The data acquisition was controlled and analyzed by X'Pert Data Collector (version 2.2d) and X'Pert Data Viewer (version 1.2c), respectively.

The X-ray diffraction pattern for the (N,N-Diethylcarbamoyl)methyl methyl (2E)but-2-ene-1,4-dioate and malic acid cocrystal is shown in FIG. 13. Unless otherwise specified, the experimental data for X-ray powder diffraction were collected at room temperature.

NMR Analysis

Proton NMR (400 MHz) NMR spectra were recorded on a Varian AS 400 NMR spectrometer equipped with an auto-sampler and data processing software. CDCl₃ (99.8% D), or MeOH-d₃ (99.8+% D), were used as solvents unless otherwise noted. The CHCl₃, or MeOH-d₃ solvent signals were used for calibration of the individual spectra. The NMR spectral pattern for the (N,N-Diethylcarbamoyl)methyl methyl (2E)but-2-ene-1,4-dioate:malic acid cocrystal is shown in FIG. 14.

### Example 6

### Synthesis, Purification and Analysis of Cocrystal of (N,N-Diethylcarbamoyl)methyl methyl (2E)but-2-ene-1,4-dioate and Citric Acid

Cocrystals of (N,N-Diethylcarbamoyl)methyl methyl (2E)but-2-ene-1,4-dioate and citric acid were prepared as follows. First, 0.25 g of citric acid was dissolved in 10 mL of ethyl acetate/heptane mixture (⅓ v/v). Then 0.50 g of (N,N-Diethylcarbamoyl)methyl methyl (2E)but-2-ene-1,4-dioate was added to the solution. The resulting suspension was briefly sonicated, and was allowed to stir for 24 hours at room temperature. The starting concentrations of (N,N-Diethylcarbamoyl)methyl methyl (2E)but-2-ene-1,4-dioate and citric acid were chosen such that the thermodynamically stable solid phase at equilibrium is the cocrystal of (N,N-Diethylcarbamoyl)methyl methyl (2E)but-2-ene-1,4-dioate and citric acid. The product was collected and dried through vacuum filtration to yield the (N,N-Diethylcarbamoyl)methyl methyl (2E)but-2-ene-1,4-dioate:citric acid (1:1) cocrystal. The measured melting points were 58° C. for (N,N-Diethylcarbamoyl)methyl methyl (2E)but-2-ene-1,4-dioate, 153° C. for citric acid, and 73° C. for the cocrystal.

Differential Scanning Calorimetry (DSC) Analysis

The DSC analysis was conducted using the TA Instruments Q2000 DSC equipped with a refrigerated cooling system. For all DSC analysis, 2-10 mg of sample was loaded into T_{zero} aluminum pans with crimpled lids. A pinhole was made at the center of the lid to avoid any pressure buildup during heating. Samples were equilibrated at 10° C. and ramped to 250° C. at a rate of 10° C. per minute under a purge of dry nitrogen gas. The data acquisition was controlled by Thermal Advantage software Release 4.9.1. The data were analyzed with Universal Analysis 2000 software (version 4.5A).

DSC thermogram in FIG. 18 shows that the (N,N-Diethylcarbamoyl)methyl methyl (2E)but-2-ene-1,4-dioate:citric acid cocrystal melts at about 73° C., which is significantly higher than the melting point of crystalline (N,N-Diethylcarbamoyl)methyl methyl (2E)but-2-ene-1,4-dioate. The melting points of (N,N-Diethylcarbamoyl)methyl methyl (2E)but-2-ene-1,4-dioate and citric acid are 58° C. and 153° C., respectively.

Thermogravimetric Analysis (TGA)

The thermal gravimetric analysis was conducted using a TA Instruments Q5000 thermogravimetric analyzer. For all TGA analysis, 5-10 mg of sample was loaded to a platinum pan and was heated to 250° C. at a rate of 10° C. per minute under a purge of dry nitrogen gas. The data acquisition was controlled by Thermal Advantage software Release 4.9.1. The data were analyzed with Universal Analysis 2000 software (version 4.5A).

The TGA thermogram shows that the (N,N-Diethylcarbamoyl)methyl methyl (2E)but-2-ene-1,4-dioate:citric acid cocrystal does not undergo any weight loss prior to melting, which indicates that the cocrystal is an anhydrous crystalline solid.

X-ray Powder Diffraction (XRPD) Analysis

Powder X-ray diffraction analysis was performed using the PANalytical X'Pert Pro X-ray diffractomer. The X-ray source was Cu K_{α} radiation (λ=1.54051 Å) with output voltage of 45 kV and current of 40 mA. The instrument adopts a para-focusing Bragg-Brentano geometry with incident divergence and scattering slits set at 1/16° and ⅛° respectively. Large Soller slit (0.04 rad) were used for both incident and diffracted beam to remove axial divergence. A small amount of powder (9-12 mg) was gently pressed onto the single crystal silicon sample holder to form a smooth surface, and samples were subjected to spinning at a rate of two revolutions per second, throughout the acquisition process. The samples were scanned from 2° to 40° in 2θ with a step size of 0.017° and a scan speed of 0.067 °/sec. The data acquisition was controlled and analyzed by X'Pert Data Collector (version 2.2d) and X'Pert Data Viewer (version 1.2c), respectively.

The X-ray diffraction pattern for the (N,N-Diethylcarbamoyl)methyl methyl (2E)but-2-ene-1,4-dioate:citric acid cocrystal is shown in FIG. 16. Unless otherwise specified, the experimental data for X-ray powder diffraction were collected at room temperature.

NMR Analysis

Proton NMR (400 MHz) NMR spectra were recorded on a Varian AS 400 NMR spectrometer equipped with an auto-sampler and data processing software. CDCl₃ (99.8% D), or MeOH-d₃ (99.8+% D), were used as solvents unless otherwise noted. The CHCl₃, or MeOH-d₃ solvent signals were used for calibration of the individual spectra. The NMR spectral pattern for the (N,N-Diethylcarbamoyl)methyl methyl (2E)but-2-ene-1,4-dioate:citric acid cocrystal is shown in FIG. 17.

Finally, it should be noted that there are alternative ways of implementing the embodiments disclosed herein. Accordingly, the present embodiments are to be considered as illustrative and not restrictive, and the claims are not to be limited to the details given herein, but may be modified within the scope and equivalents thereof.

From the foregoing description, various modifications and changes in the compositions and methods of the present disclosure will occur to those skilled in the art. All such modifications corning within the scope of the appended claims are intended to be included therein.

1. A cocrystal of (N,N-Diethylcarbamoyl)methyl methyl (2E)but-2-ene-1,4-dioate and urea.

2. The cocrystal of claim 1, having a molar ratio of (N,N-Diethylcarbamoyl)methyl methyl (2E)but-2-ene-1,4-dioate to urea of about 1:1.

3. The cocrystal of claim 1, having a DSC thermogram peak between about 75° C. and about 79° C.

4. The cocrystal of claim 1, which exhibits a characteristic scattering angle (2θ) at least at 26.3±0.2° in an X-ray powder diffractogram measured using Cu-K_{α} radiation.

5. The cocrystal of claim 1, which exhibits characteristic scattering angles (2θ) at least at 4.9±0.2°, 11.8±0.2°, 24.0±0.2°, 26.3±0.2°, and 28.2±0.2° in an X-ray powder diffraction pattern measured using Cu-K_{α} radiation.

6. The cocrystal of claim 1, which exhibits characteristic scattering angles (2θ) at least at 4.9±0.2°, 14.2±0.2°, 21.8±0.2°, 26.3±0.2°, and 31.0±0.2° in an X-ray powder diffraction pattern measured using Cu-K_{α} radiation.

7. The cocrystal of claim 1, which exhibits characteristic scattering angles (2θ) at least at 4.9±0.2°, 14.2±0.2°, 19.8±0.2°, 20.0±0.2°, 21.8±0.2°, 23.7±0.2°, 25.8±0.2°, 26.3±0.2°, 28.6±0.2°, and 31.0±0.2° in an X-ray powder diffraction pattern measured using Cu-K_{α} radiation.

8. The cocrystal of claim 1, which exhibits characteristic scattering angles (2θ) at least at 4.9±0.2°, 9.9±0.2°, 11.8±0.2°, 14.2±0.2°, 19.8±0.2°, 20.0±0.2°, 21.8±0.2°, 22.5±0.2°, 23.7±0.2°, 24.0±0.2°, 25.8±0.2°, 26.3±0.2°, 28.2±0.2°, 28.6±0.2°, and 31.0±0.2° in an X-ray powder diffraction pattern measured using Cu-K_{α} radiation.

9. A cocrystal of (N,N-Diethylcarbamoyl)methyl methyl (2E)but-2-ene-1,4-dioate and fumaric acid.

10. The cocrystal of claim 9, having a molar ratio of (N,N-Diethylcarbamoyl)methyl methyl (2E)but-2-ene-1,4-dioate to fumaric acid of about 2:1.

11. The cocrystal of claim 9, having a DSC thermogram peak between about 72° C. and about 76° C.

12. The cocrystal of claim 9, which exhibits a characteristic scattering angle (2θ) at least at 9.8±0.2° in an X-ray powder diffractogram measured using Cu-K_{α} radiation.

13. The cocrystal of claim 9, which exhibits characteristic scattering angles (2θ) at least at 9.8±0.2°, 15.1±0.2°, 25.4±0.2620 and 28.3±0.2° in an X-ray powder diffractogram measured using Cu-K_{α} radiation.

14. The cocrystal of claim 9, which exhibits characteristic scattering angles (2θ) at least at 9.8±0.2°, 15.1±0.2°, 21.5±0.2°, 22.5±0.2°, and 28.3±0.2° in an X-ray powder diffraction pattern measured using Cu-K_{α} radiation.

15. The cocrystal of claim 9, which exhibits characteristic scattering angles (2θ) at least at 7.8±0.2°, 9.8±0.2°, 12.2±0.2°, 15.1±0.2°, 21.5±0.2°, 22.5±0.2°, 23.2±0.2°, 25.4±0.2°, 26.5±0.2° and 28.3±0.2° in an X-ray powder diffraction pattern measured using Cu-K_{α} radiation.

16. The cocrystal of claim 9, which exhibits characteristic scattering angles (2θ) at least at 7.8±0.2°, 9.8±0.2°, 12.2±0.2°, 15.1±0.2°, 21.5±0.2°, 22.5±0.2°, 22.7±0.2°, 23.2±0.2°, 24.5±0.2°, 25.4±0.2 , 26.5±0.2°, 27.7±0.2°, 28.3±0.2°, 29.2±0.2°, and 34.1±0.2° in an X-ray powder diffraction pattern measured using Cu-K_{α} radiation.

17. A cocrystal of (N,N-Diethylcarbamoyl)methyl methyl (2E)but-2-ene-1,4-dioate and succinic acid.

18. The cocrystal of claim 17, having a molar ratio of (N,N-Diethylcarbamoyl)methyl methyl (2E)but-2-ene-1,4-dioate to succinic acid of about 2:1.

19. The cocrystal of claim 17, having a DSC thermogram peak between about 62° C. and about 66° C.

20. The cocrystal of claim 17, which exhibits a characteristic scattering angle (2θ) at least at 10.0±0.2° in an X-ray powder diffractogram measured using Cu-K_{α} radiation.

21. The cocrystal of claim 17, which exhibits characteristic scattering angles (2θ) at least at 7.6±0.2°, 10.0±0.2°, 14.9±0.2°, 15.3±0.2° and 28.1±0.2° in an X-ray powder diffractogram measured using Cu-K_{α} radiation,

22. The cocrystal of claim 17, which exhibits characteristic scattering angles (2θ) at least at (2θ) at least at 7.6±0.2°, 10.0±0.2°, 14.9±0.2°, 21.5±0.2° and 22.8±0.2° in an X-ray powder diffraction pattern measured using Cu-K_{α} radiation,

23. The cocrystal of claim 17, which exhibits characteristic scattering angles (2θ) at least at 7.6±0.2°, 10.0±0.2°, 12.1±0.2°, 14.9±0.2°, 15.3±0.2°, 21.5±0.2°, 22.8±0.2°, 24.3±0.2°, 27.2±0.2° and 28.1±0.2° in an X-ray powder diffraction pattern measured using Cu-K_{α} radiation.

24. The cocrystal of claim 17, which exhibits characteristic scattering angles (2θ) at least at 7.6±0.2°, 10.0±0.2°, 12.1±0.2°, 14.9±0.2°, 15.3±0.2°, 18.7±0.2°, 21.5±0.2°, 22.4±0.2°, 22.8±0.2°, 23.0±0.2°, 24.3±0.2°, 26.2±0.2°, 27.2±0.2°, and 28.1±0.2°, and 30.1±0.2° in an X-ray powder diffraction pattern measured using Cu-K_{α} radiation.

25. A cocrystal of (N,N-Diethylcarbamoyl)methyl methyl (2E)but-2-ene-1,4-dioate and maleic acid.

26. The cocrystal of claim 25, having a molar ratio of (N,N-Diethylcarbamoyl)methyl methyl (2E)but-2-ene-1,4-dioate to maleic acid of about 1:1.

27. The cocrystal of claim 25, having a DSC thermogram peak between about 65° C. and about 69° C.

28. The cocrystal of claim 25 which exhibits a characteristic scattering angle (2θ) at least at 7.7±0.2° in an X-ray powder diffractogram measured using Cu-K_{α} radiation.

29. The cocrystal of claim 25, which exhibits characteristic scattering angles (2θ) at least at 7.4±0.2°, 7.7±0.2°, 10.0±0.2°, 13.2±0.2° and 30.1±0.2° in an X-ray powder diffractogram measured using Cu-K_{α} radiation.

30. The cocrystal of claim 25, which exhibits characteristic scattering angles (2θ) at least at 7.4±0.2°, 7.7±0.2°, 10.0±0.2°, 13.2±0.2° and 20.0±0.2° in an X-ray powder diffraction pattern measured using Cu-K_{α} radiation.

31. The cocrystal of claim 25, which exhibits characteristic scattering angles (2θ) at least at 7.4±0.2°, 7.7±0.2°, 10.0±0.2°, 13.2±0.2°, 16.7±0.2°, 20.0±0.2°, 23.2±0.2°, 24.6±0.2°, 27.3±0.2° and 30.1±0.2° in an X-ray powder diffraction pattern measured using Cu-K_{α} radiation.

32. The cocrystal of claim 25, which exhibits characteristic scattering angles (2θ) at least at 7.4±0.2°, 7.7±0.2°, 10.0±0.2°, 12.7±0.2°, 13.2±0.2°, 14.9±0.2°, 16.7±0.2°, 19.6±0.2°, 20.0±0.2°, 23.2±0.2°, 24.1±0.2°, 24.6±0.2°, 27.3±0.2°, 28.1±0.2° and 30.1±0.2° in an X-ray powder diffraction pattern measured using Cu-K_{α} radiation.

33. A cocrystal of (N,N-Diethylcarbamoyl)methyl methyl (2E)but-2-ene-1,4-dioate and malic acid.

34. The cocrystal of claim 33, having a molar ratio of (N,N-Diethylcarbamoyl)methyl methyl (2E)but-2-ene-1,4-dioate to malic acid of about 1:1.

35. The cocrystal of claim 33, having a DSC thermogram peak between about 61° C. and about 65° C.

36. The cocrystal of claim 33, which exhibits a characteristic scattering angle (2θ) at least at 5.8±0.2° in an X-ray powder diffractogram measured using Cu-K_{α} radiation.

37. The cocrystal of claim 33, which exhibits characteristic scattering angles (2θ) at least at 5.8±0.2°, 14.9±0.2°, 17.5±0.2°, 22.3±0.2°, and 25.3±0.2° in an X-ray powder diffractogram measured using Cu-K_{α} radiation.

38. The cocrystal of claim 33, which exhibits characteristic scattering angles (2θ) at least at 5.8±0.2°, 10.6±0.2°, 22.3±0.2°, 23.9±0.2° and 28.3.±0.2° in an X-ray powder diffraction pattern measured using Cu-K_{α} radiation.

39. The cocrystal of claim 33, which exhibits characteristic scattering angles (2θ) at least at 5.8±0.2°, 10.6±0.2°, 11.6±0.2°, 14.9±0.2°, 17.5±0.2°, 20.0±0.2°, 21.3±0.2°, 22.3±0.2°, 23.9±0.2° and 28.3±0.2° in an X-ray powder diffraction pattern measured using Cu-K_{α} radiation.

40. The cocrystal of claim 33, which exhibits characteristic scattering angles (2θ) at least at 5.8±0.2°, 10.6±0.2°, 11.6±0.2°, 14.9±0.2°, 17.0±0.2°, 17.5±0.2 °, 18.4±0.2°, 20.0±0.2°, 20.2±0.2°, 21.3±0.2°, 22.3±0.2°, 23.9±0.2°, 25.3±0.2°, 25.4±0.2° and 28.3±0.2° in an X-ray powder diffraction pattern measured using Cu-K_{α} radiation.

41. A cocrystal of (N,N-Diethylcarbamoyl)methyl methyl (2E)but-2-ene-1,4-dioate and citric acid.

42. The cocrystal of claim 41, having a molar ratio of (N,N-Diethylcarbamoyl)methyl methyl (2E)but-2-ene-1,4-dioate to citric acid of about 1:1.

43. The cocrystal of claim 41, having a DSC thermogram peak between about 71° C. and about 75° C.

44. The cocrystal of claim 41, which exhibits a characteristic scattering angle (2θ) at least at 6.2±0.2° in an X-ray powder diffractogram measured using Cu-K_{α} radiation.

45. The cocrystal of claim 41, which exhibits characteristic scattering angles (2θ) at least at 6.2±0.2°, 9.8±0.2°, 13.5±0.2°, and 18.6±0.2° in an X-ray powder diffractogram measured using Cu-K_{α} radiation.

46. The cocrystal of claim 41, which exhibits characteristic scattering angles (2θ) at least at 6.2±0.2°, 6.9±0.2°, 18.9±0.2°, 19.1±0.2° and 25.0±0.2° in an X-ray powder diffraction pattern measured using Cu-K_{α} radiation.

47. The cocrystal of claim 41, which exhibits characteristic scattering angles (2θ) at least at 6.2±0.2°, 6.9±0.2°, 12.3±0.2°, 13.4±0.2 , 13.9±0.2°, 18.6±0.2°. 19.1±0.2°, 25.0±0.2°, 26.4±0.2° and 31.2±0.2° in an X-ray powder diffraction pattern measured using Cu-K_{α} radiation.

48. The cocrystal of claim 41, which exhibits characteristic scattering angles (2θ) at least at 6.2±0.2°, 6.9±0.2°, 9.8±0.2°, 12.3±0.2°, 13.4±0.2°, 13.9±0.2°, 18.6±0.2°, 19.1±0.2°, 20.6±0.2°, 21.9±0.2°, 24.8±0.2°, 25.0±0.2°, 26.4±0.2°, 30.0±0.2° and 31.2±0.2° in an X-ray powder diffraction pattern measured using Cu-K_{α} radiation.

49 A pharmaceutical composition comprising a cocrystal according to claim 1 and a pharmaceutically acceptable carrier.

50. A pharmaceutical composition comprising a cocrystal according to claim 9 and a pharmaceutically acceptable carrier.

51. A pharmaceutical composition comprising a cocrystal according to claim 17 and a pharmaceutically acceptable carrier.

52. A pharmaceutical composition comprising a cocrystal according to claim 25 and a pharmaceutically acceptable carrier.

53. A pharmaceutical composition comprising a cocrystal according to claim 33 and a pharmaceutically acceptable carrier.

54. A pharmaceutical composition comprising a cocrystal according to claim 41 and a pharmaceutically acceptable carrier.

55. A method of treating a disease in a patient in need of such treatment, comprising administering to the patient a therapeutically effective amount of a cocrystal according to claim 1.

56. A method of treating a disease in a patient in need of such treatment, comprising administering to the patient a therapeutically effective amount of a cocrystal according to claim 9.

57. A method of treating a disease in a patient in need of such treatment, comprising administering to the patient a therapeutically effective amount of a cocrystal according to claim 17.

58. A method of treating a disease in a patient in need of such treatment, comprising administering to the patient a therapeutically effective amount of a cocrystal according to claim 25.

59. A method of treating a disease in a patient in need of such treatment, comprising administering to the patient a therapeutically effective amount of a cocrystal according to claim 33.

60. A method of treating a disease in a patient in need of such treatment, comprising administering to the patient a therapeutically effective amount of a cocrystal according to claim 41.

61. The method of claim 55, wherein the disease is selected from the group consisting of inflammatory arthritis, inflammatory bowel disease, asthma, chronic obstructive pulmonary disease and neurodegenerative disorders.

62. The method of claim 55, wherein the disease is multiple sclerosis.

63. The method of claim 55, wherein the disease is psoriasis.
